(19) United States
(12) Patent Application Publication
(10) Pub. No.: US 2025/0169528 A1
(43) Pub. Date: May 29, 2025

(54) Fireproofing Agents

(71) Applicant: Symrise AG, Holzminden (DE)

(72) Inventors: Jenny Weissbrodt, Holzminden (DE); Halil El-Wayss, Stadtoldendorf (DE); Daniel Balke, Höxter (DE)

(21) Appl. No.: 18/851,358

(22) PCT Filed: Mar. 28, 2023
(86) PCT No.: PCT/EP2023/058030

§ 371 Date: Sep. 26, 2024

(30) Foreign Application Priority Data
Mar. 28, 2022 (WO) PCT/EP2022/058056

(51) Int. Cl. A23P 10/40; A23L 27/00; A23L 27/10; A23L 29/00; A23P 10/22; A23P 10/30

(52) U.S. Cl. CPC A23P 10/40; A23L 27/10; A23L 27/72; A23L 29/04; A23P 10/22; A23P 10/30

(57) ABSTRACT

The present invention relates to a powdered composition comprising or consisting of either a spray-drying product, or alternatively, a composition comprising or consisting of at least one core-shell microcapsule, and optionally a carrier system, and further comprising or consisting of an effective amount of a fireproofing agent selected from the group consisting of polysaccharides, sugar alcohols, proteins, water-soluble and/or water-insoluble dietary fibres, plant-based oils, edible mineral salts such as sodium chloride or calcium chloride, salts of organic acids such as calcium lactate or magnesium stearate, plant extracts such as herbal extracts and vegetable extracts, for examples tea extracts, or mixtures thereof. Moreover, the present invention relates to a method for the preparation of a corresponding powdered composition, a corresponding powdered composition as such, and its use for the preparation of consumer products as well as consumer products as such comprising the powdered composition according to the invention. Additionally, the present invention relates to the use of the above-mentioned fireproofing agents for the preparation of said powdered composition and for reducing its combustion potential.

### FIELD OF THE INVENTION

The present invention relates to a powdered composition comprising or consisting of either a spray-drying product, or alternatively, a composition comprising or consisting of at least one core-shell microcapsule, and optionally a carrier system, and further comprising or consisting of an effective amount of a fireproofing agent selected from the group consisting of polysaccharides, sugar alcohols, proteins, water-soluble and/or water-insoluble dietary fibres, plant-based oils, edible mineral salts such as sodium chloride or calcium chloride, salts of organic acids such as calcium lactate or magnesium stearate, plant extracts such as herbal extracts and vegetable extracts, for example tea extracts, or mixtures thereof. Moreover, the present invention relates to a method for the preparation of a corresponding powdered composition, a corresponding powdered composition as such, and its use for the preparation of consumer products as well as consumer products as such comprising the powdered composition according to the invention. Additionally, the present invention relates to the use of the above-mentioned fireproofing agents for the preparation of said powdered composition and for reducing its combustion potential.

### BACKGROUND OF THE INVENTION

Many encapsulated flavours, but also other active ingredients, are provided or processed in the form of powders. These powders can be flammable, ignitable, or explosive depending on their particle size and structure, their water-content as well as their composition. But other powdered products or compositions used in the industry are also combustible. Particular danger is given when fine powders are whirled up so that the combustible dust is suspended in the air. In this case, even a small amount of energy is usually sufficient to ignite the dust/air-mixture.

Little is known about the exact relationships between explosion characteristics and powder composition and structure. It is only clearly known that the particle size has an influence on the explosion characteristics. The smaller the particles the more rapid and violent the burning. More detailed knowledge about the influence of the composition is not available, since mostly only single substances have been tested and their values published so far (e.g. GESTIS database).

However, the burning behaviour of a substance, composition or product formulation is a crucial parameter in particular in view of plant safety concepts as well as the transportation of powdered goods. For example, various active ingredients such as flavours and fragrances are usually incorporated into product formulations as microparticles either in encapsulated form (as common core-shell microcapsules) or as spray-drying products whereby the active is freely dispersed within a matrix (i.e. not in the form of core-shell microcapsules) in order to prepare the flavoured or perfumed consumer product formulations.

Thereby spray-drying is based on the preparation of a dry powder from a liquid or slurry by rapidly drying with a hot gas using a spray nozzle operated at high pressures to disperse the liquid or slurry to obtain fine particles (microparticles) with homogeneous particle sizes. Based on this process a broad variety of consumer products is prepared such as milk powder, coffee, flavouring, dry beverage preparations, etc.

Other active ingredients such as volatile aromas and flavours are usually provided in encapsulated form (microcapsules) which can be incorporated into a variety of different product formulations allowing for a targeted release of the active ingredients. Said microcapsules are usually present in the form of dispersions but also can be dried to form a powdered composition which then can be incorporated into a variety of different powdered formulations. Such core-shell microcapsules (in the following “microcapsules”) can be formed by interfacial polycondensation, interfacial cross-linking or polymerization, in situ polymerization or matrix polymerization e.g. by spray-drying, wherein the microparticle is formed by evaporation of matrix solvent.

Considering the small particle size of such powdered formulations and the properties of the active ingredients as well as the high processing temperatures, such microparticles may be considered dangerous materials, especially if they contain volatile and/or flammable ingredients such as flavours or perfumes requiring increased safety precautions during manufacturing, processing, storage, and transportation.

According to the Globally Harmonized System of Classification and Labelling of Chemicals (GHS) in terms of physical hazards, flammable solids are defined as solids which are readily combustible or may cause or contribute to fire through friction, while readily combustible solids are defined as powdered, granular, or pasty substances which are dangerous if they can be easily ignited by brief contact with an ignition source, such as a burning match, and if the flame spreads rapidly based on the results of the burning rate test according to test method N.1 as described in Part III, sub-section 33.2.1 of the UN ST/SG/AC.10. In case of burning rates of >2.2 mm/s or burning times of <45 s, respectively, the corresponding tested substances or compositions are characterized as flammable and thus as physical hazards requiring additional safety precautions.

Such substances or compositions are classified according to the HAZMAT class 4.1 as flammable solids for transportation purposes and thus as dangerous goods or materials, i.e. substances or compositions that, e.g. when transported, are a risk to health, safety, property or the environment due to their nature, their physical or chemical properties or their condition during transport.

This leads to additional costs as specific labelling and safety precautions are required in production as well as during transport. To avoid the additional requirements, the burning rate of the powders must be reduced to ≤2.22 mm/s (≤100 mm in 45 s).

There are some approaches for reducing the burning rate of a substance or composition, e.g. by increasing the particle size e.g. by agglomeration and thus reduction of the reactive surface. However, bigger particles are not suitable for all applications.

However, only few data are available on how the explosion characteristics can be influenced by the addition of specific substances. Additionally, only few solutions have been provided up to now in order to make such powdered products less harmful.

Substance or compounds are characterized by the burning rate (r), a measure of its stationary linear combustion rate which is measured in length over time. Thereby the burning rate is influenced by the following parameters, among others: the chemical composition, the surface characteristics, the temperature, the pressure etc.

In addition, the burning number (or combustion number) is a criterion for the spread of a fire after local and external ignition of dust samples.

Moreover, the minimal ignition energy (MIE), i.e. is a further safety-related parameter. It describes the ignition sensitivity of a substance to discharges of static electricity. Said parameter must be known in order to apply the explosion protection measure ‘avoidance of effective ignition sources’.

While the burning rate is primarily important for the transport of the substance, the minimum ignition energy is elementary for all processes of production, packaging, dosing, transport and further processing.

U.S. Pat. No. 7,204,998 B2 describes perfuming or flavouring microcapsules that include a fireproofing agent, wherein said fireproofing agent is selected from the group consisting of sodium silicate, potassium silicate, monoammonium phosphate or carbonate, diammonium phosphate, mono-, di- or trisodium phosphate, sodium hypophosphite, melamine cyanurate, and mixtures thereof. However, said substances such as sodium silicate, potassium silicate, monoammonium phosphate or carbonate are no suitable food additives and are usually used as adhesives, detergent auxiliaries, or fertilizers. Moreover, melamine cyanurate is considered to be toxic and leads to acute kidney injury. Thus, these fireproofing agents proposed by U.S. Pat. No. 7,204,998 B2 are not suitable in the preparation of (powdered) consumer products such as foodstuff or cosmetic products or the corresponding powdered ingredients or components thereof.

WO 2019/170528 A1 suggests the addition of talc as fireproofing agent to powdered compositions. However, these powdered compositions require a high amount of talc in order to improve the minimal ignition temperature (MIE) of said powdered compositions. Additionally, it is known that the inhalation of fine talc powder can lead to inflammations in the peripheral respiratory tract and might cause cancer or other types of disease, which is why the use of talc has recently come under increased criticism.

Therefore, new approaches are needed to make such powdered products “less dangerous” e.g., for handling, storage, or transportation, by specifically incorporating fire- or explosion-retardant substances into the product formulations. In particular, new fire- or explosion-retardant additives are required.

High demands must be placed on the fire- or explosion-retardant substances in question: They must be harmless to health, environmentally friendly and preferably also neutral in taste, for example when used in food preparations. In addition, the added fire- or explosion-retardant substances must not significantly change the properties of the formulations to which they are added (odour, colour, stability etc.) and must ensure stable incorporation into the underlying product formulations.

Thus, there is need to improve these substances or compositions or product formulations with regard to their burning and explosion behaviour, in particular, without negatively influencing the underlying properties of the composition or product formulation. Simultaneously, the final compositions should be food and health safe.

### SUMMARY OF THE INVENTION

The present invention was made in view of the drawbacks of the prior art described above, and the object of the present invention is to provide a powdered composition comprising or consisting of either a spray-drying product, or alternatively, a powdered composition comprising or consisting of at least one microcapsule, and optionally a carrier system, and further comprising or consisting of an effective amount of a fireproofing agent as specified herein which shows a reduced burning number and/or burning rate allowing for a facilitated handling, storage and transportation with reduced costs in terms of required protective measures.

Accordingly, the primary aim of the present invention is therefore to provide a powdered composition comprising a fireproofing agent as specified herein which allows for a safer handling of said powdered composition and which are simultaneously food safe.

To solve the problem, the present invention provides specific fire- or explosion-retardant substances which are food-safe and are selected from the group of foodstuffs, food additives and food processing aids and which simultaneously allow the explosion characteristics of flavour powders to be lowered in a targeted manner.

Thus, in a first aspect the present invention relates to a powdered composition comprising or consisting of either
a spray-drying product, or alternatively,
a composition comprising or consisting of at least one microcapsule,
and optionally a carrier system,
   further comprising or consisting of an effective amount of a fireproofing agent selected from the group consisting of polysaccharides, sugar alcohols, proteins, water-soluble and/or water-insoluble dietary fibres, plant-based oils, edible mineral salts such as sodium chloride or calcium chloride, salts of organic acids such as calcium lactate or magnesium stearate, plant extracts such as herbal extracts and vegetable extracts (e.g. tea extracts), or mixtures thereof.

According to a second aspect the present invention relates to a method for the preparation of a powdered composition, comprising the following steps:
(a) providing an aqueous dispersion, optionally comprising at least one microcapsules, comprising a fireproofing agent selected from the group consisting of polysaccharides, sugar alcohols, proteins, water-soluble and/or water-insoluble dietary fibres, plant-based oils, edible mineral salts such as sodium chloride or calcium chloride, salts of organic acids such as calcium lactate or magnesium stearate, plant and vegetable extracts such as tea extracts, or mixtures thereof, and
(b) spray-drying the dispersion of step (a) to obtain a powdered composition.

Additionally, in a further aspect the present invention relates to powdered compositions prepared according to the inventive method, wherein the powdered composition comprises ≤6% by weight and preferably ≤5% by weight of (remaining) water based on the total weight of the powdered composition.

In a further aspect the present invention relates to the use of a fireproofing agent selected from the group consisting of polysaccharides, sugar alcohols, proteins, water-soluble and/or water-insoluble dietary fibres, plant-based oils, edible mineral salts such as sodium chloride or calcium chloride, salts of organic acids such as calcium lactate or magnesium stearate, plant extracts such as herbal extracts and vegetable extracts (e.g. tea extracts), or mixtures thereof, for the preparation of a powdered composition according to the invention and/or for reducing the combustion potential of a powdered composition.

Finally, the present invention relates to the use of a powdered composition according to the invention for the preparation of consumer products as well as to the consumer products as such comprising or consisting of the powdered composition according to the present invention.

Based on the use/incorporation of an effective amount of the fireproofing agents according to the present invention it is possible to achieve burning rates of ≤2.22 mm/s for various powdered formulations and a significant improvement of the minimum ignition energy (MIE). Based thereon potential risks of powdered compositions and formulations can efficiently be reduced, resulting in a mitigation of dangerous goods, and thus reduced transport costs and costs for additional safety precautions during manufacture, processing, storage and transportation. In addition, it was surprisingly found that said fireproofing agents are harmless to health, environmentally friendly and also neutral in taste: Moreover, the added fire- or explosion-retardant substances do not significantly change the underlying properties of the formulations to which they are added such as their odour, colour or stability can stably be incorporated into various consumer product formulations.

Surprisingly, it was found that carbohydrates, i.e. biomolecules consisting of carbon, hydrogen and oxygen atoms are excellent fireproofing agents.

The invention is specified in the appended claims. The invention itself, and its preferred variants, other objects and advantages, are however also apparent from the following detailed description in conjunction with the accompanying examples and figures.

### BRIEF DESCRIPTION OF DRAWINGS

FIG. 1 is a representation of the test procedure for determining the minimal ignition energy (MIE).

FIG. 2 is a schematic representation of the preparation of powdered compositions.

FIG. 3 is a schematic representation of the particle formation in a spray-drying process, i.e. the spray-drying product preparation of powdered compositions according to the invention.

FIG. 4 is a diagram showing the dust index of spray-dried beverage formulations or aroma preparations (“peach mix” or “peach flavour”) comprising different potential fireproofing agents.

FIG. 5 is a diagram showing the dust index and the flow index of spray-dried beverage formulations or aroma preparations (“peach mix” or “peach flavour”) comprising different potential fireproofing agents.

FIG. 6 shows the results of the thermogravimetric analysis.

FIG. 7 is a schematic representation of different pore types.

### DETAILED DESCRIPTION OF THE INVENTION

Hereinafter, the present invention is described in more detail.

Surprisingly, it was found that various dietary fibres and other food additives such as salts are highly suitable in improving the safety of powdered substances and compositions.

In a first aspect, the present invention relates a powdered composition comprising or consisting of either
a spray-drying product, or alternatively,
a composition comprising or consisting of at least one microcapsule, and
optionally a carrier system,
   further comprising or consisting of an effective amount of a fireproofing agent selected from the group consisting of polysaccharides and derivatives thereof, sugar alcohols, proteins, water-soluble and/or water-insoluble dietary fibres, plant-based oils and fats, mineral salts such as sodium chloride or calcium chloride, salts of organic acids such as calcium lactate or magnesium stearate, plant extracts such as tea extracts, or mixtures thereof.

### Polysaccharides and Derivatives Thereof:

The term “polysaccharide” refers to substances being based on chains of carbohydrates composed of monosaccharides linked by glycosidic bonds, wherein the chain comprises or consists of more than ten monosaccharides of the general formula (CH₂O)ₙ wherein n is three or more. Examples of carbohydrates include mono- and disaccharides, glucose, galactose, mannose, lactose, maltose, and sucrose; fructose and mannose; polysaccharides such as starches.

Suitable fireproofing agents according to the present invention are polysaccharides and derivatives thereof, such as starch, modified starch, glycogen, and cellulose, chitin, xanthan gum, exsudate gums such as gum arabic, gum karya, and gum tragacanth, agar-agar, alginates or tylosin, callose or laminarin, chrysolaminarin, xylan, arabinoxylan, mannan, fucoidan and galactomannan, chitosan, or mixtures thereof.

Further suitable gums are natural gums such as seaweed extracts (e.g. alginates), plant exudates (e.g. arabic and tragacanth gums), gums from seed or root (e.g. potato starch), and gums obtained by microbial fermentation (e.g. gum xanthan) and modified gums such as cellulose and starch derivatives, such as ethers and esters of cellulose.

### Sugar Alcohols:

Sugar alcohols are organic compounds belonging to the group of polyhydric alcohols, i.e. polyols, which are typically derived from natural carbohydrates and which are naturally present in various fruits and vegetables. Sugar alcohols such as mannitol or sorbitol are used widely in the food industry as food additives such as thickeners and sweeteners. Due to their reduced sweetness, their advantages with regard to diet control and dental health, and the fact that they are usually well tolerated, such sugar alcohols can be advantageously incorporated into a variety of product formulations as fireproofing agents according to the present invention, such as food product formulations.

Suitable sugar alcohols within the context of the present invention are preferably naturally occurring sugar alcohols and are preferably selected from the group consisting of glycerol, erythritol, threitol, arabitol, ribitol, xylitol, sorbitol, mannitol, maltitol, isomalt, isomaltitol, dulcitol, lactitol, and the physiologically acceptable salts of these sugar alcohols, especially their sodium salts, potassium salts, calcium salts or ammonium salts, as well as mixtures of the afore-mentioned sugar alcohols and/or their salts.

### Proteins and Derivatives Thereof:

The protein used within the context of the present invention may be any protein and/or amino acid mixture known to be suitable for use in consumer products such as nutritional products, cosmetic products, pharmaceutical products, homecare products, textile care products and the like. Typical proteins are animal proteins, vegetable proteins such as soy protein, milk proteins such as skimmed milk protein, whey protein and casein, and amino acids (or salts thereof) such as isoleucine, phenylalanine, leucine, lysine, methionine, threonine, tryptophan, arginine, glutamine, taurine, valine. Further preferred protein sources are whey protein, sodium caseinate or calcium caseinate, optionally with amino acids added. For some applications, a preferred protein source is hydrolysed protein (protein hydrolysate) which may or may not have amino acids added.

The protein hydrolysate may be any suitable protein hydrolysate used in a nutritional composition, such as soy protein hydrolysate, casein hydrolysate, whey protein hydrolysate, other animal and plant protein hydrolysates, and mixtures thereof. The protein hydrolysate of the composition is preferably a soy protein hydrolysate, a whey protein hydrolysate or a casein protein hydrolysate comprising short peptides and amino acids, optionally with additional amino acids added. In a preferred embodiment, the suitable protein hydrolysate contains a high proportion of free amino acids (e.g. more than 40% by weight) and low molecular weight peptide fragments.

The hydrolysed protein is also preferably spiked with various free amino acids to provide a nutritionally balanced amino acid content. Examples of such free amino acids include, but are not limited to, L-tryptophan, L-methionine, L-cystine, L-tyrosine and L-arginine.

Preferably, the proteins used within the context of the present invention are commercially or naturally available animal proteins or plant proteins as well as mixtures thereof, such as soy protein, milk proteins, whey protein and the like as well as the corresponding animal and plant protein hydrolysates and derivatives thereof.

According to a preferred embodiment the powdered composition comprises or consists of from 1% to 80% by weight, and preferably from 5% to 60% by weight, and even more preferred from 10% to 40% by weight and most preferred from 2.5% to 30% by weight of the protein, based on the total weight of the powdered composition.

### Plant-Based Oils and Fats:

The fat ingredient may be any lipid or fat known to be suitable for use in consumer products such as nutritional products, cosmetic products, pharmaceutical products, homecare products, textile care products and the like. Typical fats include, but are not limited to, safflower oil, canola oil, olive oil, cottonseed oil, coconut oil, palm oil, palm kernel oil, soybean oil, sunflower oil, and fractions of any protruding oils derived therefrom, such as palm olein, medium chain triglycerides (MCTs), and fatty acid esters, wherein the fatty acids are, for example. e.g. arachidonic acid, linoleic acid, palmitic acid, stearic acid, docosahexaenoic acid, eicosapentaenoic acid, linolenic acid, oleic acid, lauric acid, capric acid, caprylic acid, caproic acid. High oleic acid forms of various oils are also considered suitable for the present use, such as high oleic acid sunflower oil and high oleic acid safflower oil.

Preferably, the fat-based fireproofing component used within the context of the present invention is a plant-based oil (vegetable oil) or fat, i.e. a triglyceride extracted from plants, and even more preferred an edible or food-grade vegetable oil, such as canola oil, soybean oil, safflower oil, peanut oil, cottonseed oil, sunflower oil, avocado oil, sesame oil, olive oil, flax oil, coconut oil, palm oil, corn oil, rapeseed oil, hemp seed oil, and other nut oils such as walnut oil, almond oil, beech nut oil, Brazil nut oil, cashew oil, hazelnut oil, macadamia oil, mongongo nut oil, pecan oil, pine nut oil, pistachio oil, pumpkin seed oil, as well as citrus oils such as grapefruit seed oil, lemon oil or orange oil and oils from melons and gourd seeds such as bitter gourd oil, watermelon seed oil and the like. Further suitable oils or fats are selected from the following list: bottle gourd oil, buffalo gourd oil, Balanos oil, bladderpod oil, Burdock oil, Buriti oil, butternut squash seed oil, candlenut oil, carrot seed oil, egusi seed oil, açai oil, artichoke oil, astrocaryum murumuru butter, black seed oil, blackcurrant seed oil, borage seed oil, evening primrose oil, flaxseed oil, amaranth oil, apricot oil, apple seed oil, argan oil, babassu oil, Ben oil, Borneo tallow nut oil, cape chestnut oil, carob pod oil, castor oil, cocoa butter, cocklebur oil, cohune oil, coriander seed oil, date seed oil, dika oil, false flax oil made of the seeds of Camelina sativa, grape seed oil, hemp oil, jojoba oil, kapok seed oil, kenaf seed oil, lallemantia oil, mafura oil, mango oil, marula oil, meadowfoam seed oil, mustard oil, neem oil, Niger seed oil, nutmeg butter, okra seed oil, papaya seed oil, perilla seed oil, persimmon seed oil, pequi oil, pili nut oil, pomegranate seed oil, poppyseed oil, pracaxi oil, prune kernel oil, quinoa oil, radish oil, ramtil oil, rice bran oil, Royle oil, Sacha inchi oil, Salicornia oil, sapote oil, seje oil, shea butter, taramira oil, tea seed oil, thistle oil, tigernut oil, tobacco seed oil, tomato seed oil, tung oil, wheat germ oil, Illipe butter, mowrah butter, Passiflora edulis passion fruit oil, rose hip seed oil, rubber seed oil, tamanu or foraha oil, tonka bean oil.

### (Edible) Mineral Salts:

Further suitable fireproofing agents are selected from the group of mineral/inorganic salts and preferably, edible mineral salts such as sodium chloride (NaCl) or calcium chloride (CaCl₂)).

The term “mineral salts” used herein refers to inorganic chemical compounds consisting of an ionic assembly of positively charged cations and negatively charged anions. In inorganic salts, the cations are usually formed by metals and the anions are usually formed by non-metals or their oxides. In the narrowest sense, the term “salt” refers to the compound sodium chloride (NaCl, i.e. table salt), and in a broader sense, to all compounds made up of anions and cations bas on ionic bonds.

Suitable fireproofing agents from the group of edible mineral salts are for example sodium chloride or calcium chloride. Such salt-based fireproofing agents are particularly suitable for use in dry food preparations and in particular food preparations requiring a salty taste such as salted foods, pickled foods, and snack foods.

Further suitable mineral salts which can suitably be incorporated in powdered compositions for consumer products such as cosmetic products, pharmaceutical products, homecare products, textile care products and the like are amongst others: magnesium chloride (MgCl₂), sodium carbonate (Na₂CO₃), sodium bicarbonate (NaHCO₃), potassium chloride (KCl), sodium sulfate (Na₂SO₄), calcium carbonate (CaCO₃), and calcium phosphate (Ca₃(PO₄)₂) and the like as well as mixtures of the afore-mentioned substances.

According to a preferred embodiment, the salt-based fireproofing agent according to the present invention is advantageously selected from minerals salts of the group consisting of: magnesium chloride (MgCl₂), potassium chloride (KCl), sodium sulfate (Na₂SO₄), and calcium phosphate (Ca₃(PO₄)₂) as well as mixtures of the afore-mentioned substances.

According to a preferred embodiment the powdered composition comprises or consists of from 1% to 80% by weight, and preferably from 5% to 60% by weight, and even more preferred from 10% to 40% by weight and most preferred from 2.5% to 20% by weight of the salt, based on the total weight of the powdered composition.

### Salts of Organic Acids:

The salts of organic acids according to the present invention furthermore include the salts of barium, lithium, sodium, zinc, bismuth, chromium, cobalt, copper, potassium, strontium, titanium, tungsten, magnesium, caesium, iron, nickel, silver, aluminium, tin, or calcium of organic acids, and in particular the corresponding salts of saturated and/or unsaturated organic acids such as fatty acids, e.g. stearic acid, behenic acid, erucic acid, oleic acid, linoleic acid or dimerized derivatives thereof as well as other derivatives thereof.

Preferably, according to the present invention salts of long-chained organic acids, and in particular preferably long-chain fatty acids, are used as the fireproofing component.

According to a particularly preferred embodiment the fireproofing agent is the salt of a saturated and/or unsaturated (monounsaturated or polyunsaturated) organic acid, and preferably fatty acid, having a chain length of 13 or more carbon atoms.

Fatty acids differ in the number of carbon atoms and thus their chain lengths. Based on their chain lengths, fatty acids can be classified as lower or short-chain fatty acids (having chain lengths of up to 6-8 carbon atoms), middle or medium-chain fatty acids (having chain lengths of 6-8 to 12 carbon atoms), higher or long-chain fatty acids (having chain lengths of 13 to 21 carbon atoms), and very long-chain fatty acids (having chain lengths of more than 22 carbon atoms).

Said fatty acids are aliphatic in nature and either linear or branched, and preferably linear within the context of the present invention.

According to the present invention particularly suitable salts of saturated fatty acids are for example the salts of the following saturated fatty acids: tridecylic acid, myristic acid, pentadecylic acid, palmitic acid, margaric acid, stearic acid, nonadecylic acid, arachidic acid, heneicosylic acid, behenic acid, tricosylic acid, lignoceric acid, pentacosylic acid, cerotic acid, carboceric acid, montanic acid, nonacosylic acid, melissic acid, hentriacontylic acid, lacceroic acid, psyllic acid, geddic acid, ceroplastic acid, hexatriacontylic acid, heptatriacontylic acid, octatriacontylic acid, nonatriacontylic acid, tetracontylic acid, and mixtures thereof.

According to the present invention suitable salts of unsaturated (monounsaturated or polyunsaturated) fatty acids are for example the salts of the following unsaturated fatty acids: mono-unsaturated fatty acids (such as myristoleic, palmitoleic acid, sapienic acid, oleic acid, elaidic acid, vaccenic acid, gadoleic acid, eicosenoic acid, erucic acid, nervonic acid), di-unsaturated fatty acids (such as linoleic acid, eicosadienoic acid, docosadienoic acid), tri-unsaturated fatty acids (such as linolenic acid, pinolenic acid, eleostearic acid, mead acid, dihomo-γ-linolenic acid, eicosatrienoic acid), tetra-unsaturated fatty acids (such as stearidonic acid, arachidonic acid, eicosatetraenoic acid, adrenic acid), pentaunsaturated fatty acids (such as bosseopentaenoic acid, eicosapentaenoic acid, ozubondo acid, sardine acid, tetracosanolpentaenoic acid), hexa-unsaturated fatty acids (such as cervonic acid, herring acid), and mixtures thereof.

Most preferred the salts used as fireproofing agents according to the present invention are magnesium, calcium, potassium, sodium, and/or ammonium salts of the afore-mentioned (un) saturated fatty acids having chain lengths of 13 or more carbon atoms.

According to another preferred embodiment the fireproofing agent is the salt of a saturated and/or unsaturated (monounsaturated or polyunsaturated) monocarboxylic acid, dicarboxylic acid or tricarboxylic acid each having a chain length of 13 or more carbon atoms.

According to a preferred variant, the fireproofing agent according to the present invention is the salt of a saturated (preferably linear) monocarboxylic acid having a chain length of 13 or more carbon atoms with the general formula CₙH₂ₙ₊₁COOH or CₙH₂ₙO₂ with n≥12 such as for example: tridecylic acid, myristic acid, pentadecylic acid, palmitic acid, margaric acid, stearic acid, nonadecylic acid, arachidic acid, heneicosanoic acid, behenic acid, tricosanoic acid, lignoceric acid, pentacosanoic acid, cerotinic acid, carboceric acid, montanic acid, nonacosanoic acid, melissic acid, hentriacontanoic acid, lacceric acid, psyllic acid, geddic acid, ceroplastinic acid, hexatriacontanoic acid, heptatriacontanoic acid, octatriacontanoic acid, nonatriacontanoic acid, tetracontanoic acid, and mixtures thereof.

According to a preferred variant, the fireproofing agent according to the present invention is the salt of an unsaturated (preferably linear) monocarboxylic acid having a chain length of 13 or more carbon atoms.

According to another preferred variant, the fireproofing agent according to the present invention is the salt of a saturated (preferably linear) dicarboxylic acid having a chain length of 13 or more carbon atoms with the general formula HOOC—(CH₂)ₙ—COOH, with n≥10 such as for example: brassylic acid, tetradecanedioic acid, pentadecanedioic acid, thapsic acid, and mixtures thereof.

According to another preferred variant, the fireproofing agent according to the present invention is the salt of an unsaturated (preferably linear) dicarboxylic acid having a chain length of 13 or more carbon atoms.

According to a further preferred variant, the fireproofing agent according to the present invention is the salt of a branched saturated tricarboxylic acid having a chain length of 13 or more carbon atoms.

According to a further preferred variant, the fireproofing agent according to the present invention is the salt of a branched unsaturated tricarboxylic acid having a chain length of 13 or more carbon atoms.

According to an even more preferred embodiment the fireproofing agent according to the present invention is magnesium stearate.

### Plant Extracts Such as Herbal and Vegetable Extracts:

According to another preferred variant, the fire proofing agent is a plant extract such as an herbal extract, fruit extract and/or vegetable extract which are obtained from different parts of the plant and which are conventionally prepared by extraction of the whole plant, but also in individual cases exclusively from blossom and/or leaves, wood, bark or roots of the plant. The inventive plant extracts can be obtained from the corresponding fresh or dried plants or plant parts. Plant extracts, specific highly active plant extract fractions and highly pure active substances isolated from plant extracts can be employed.

Suitable plant extracts can be any of the plant extracts suitable for foodstuffs or other consumer products such as cosmetic products, pharmaceutical products, homecare products, textile care products and the like. Mixtures of two or more plant extracts can also be employed. The plant extracts can be used in both pure and diluted form.

Particularly advantageous for cosmetic, dermatological, pharmaceutical and/or oral hygiene purposes are the extracts of aloe, witch hazel, algae, oak bark, willowherb, stinging nettle, dead-nettle, butcher's broom, hops, chamomile, yarrow, amica, calendula, burdock, horsetail, whitethorn, rose, lime blossom, liquorice, almond, pine, horse chestnut, sandalwood, juniper, coconut, mango, apricot, orange, lemon, lime, grapefruit, apple, strawberry, raspberry, grape, pomegranate, green tea, rooibos, honeybush, grapefruit seed, kiwi, avocado, cucumber, wheat, oats, barley, sage, thyme, wild thyme, lavender, rosemary, peppermint, melissa, birch, elder, olive, mallow, lady's smock, horsetail, willow bark, restharrow, coltsfoot, marshmallow, ginseng, ginkgo, Pueraria, sophora, honeysuckle, angelica root, cinnamon, lemongrass and ginger root. The extracts from aloe vera, chamomile, algae, rosemary, calendula, ginseng, cucumber, sage, stinging nettle, lime blossom, arnica and witch hazel are particularly preferred here.

### Dietary Fibres:

Dietary fibres are largely indigestible food components and defined as the portion of plant-derived food that cannot be completely broken down by human digestive enzymes, i.e. the term “dietary fibre” is commonly understood to refer to a component of food which is non-digestible and non-metabolizable by humans. More specifically, “dietary fibres” are defined as edible carbohydrate polymers containing three or more monomeric units which are neither digested nor absorbed in the small intestine of humans and which either occur naturally in food, which are obtained by physical, enzymatic, or chemical means from raw food materials, or which been shown by generally accepted scientific evidence to have a beneficial physiological effect.

Thereby, water-soluble dietary fibres, i.e. soluble fibres that readily dissolve in water, are distinguished from water-insoluble dietary fibres. For the purposes of this invention the commonly known definitions of these two types of dietary fibre will be used. Therefore, the term “insoluble dietary fibre” refers to insoluble, substantially non-swellable dietary fires while the term “soluble dietary fibre” means dietary fibre which is water soluble or water swellable. Most dietary fibres are neutral in taste, colour and odour and thus can advantageously be incorporated into a variety of consumer product formulations such as food products or cosmetic products without negatively influencing the product properties. In addition, most of the commonly used dietary fibres are well tolerated. The term “dietary fibre” refers to soluble, insoluble, fermentable, non-fermentable or any combination of such dietary fibre. The dietary fibre may be, for example, soya fibre, pectin, certain resistant starches, oligofructose, inulins, oat fibre, pea fibre, guar gum, gum acacia, modified cellulose and the like.

Suitable dietary fibres can be selected, for example, from soluble and insoluble dietary fibres. Non-limiting examples of dietary fibres can include, but are not limited to dietary fibres that are insoluble under physiological conditions, such as: β-glucans, cellulose, modified cellulose such as microcrystalline cellulose (MCC), cellulose ethers, hemicelluloses, lichenin, chitin, chitosan, lignins, xanthan gum, plant fibres, in particular cereal fibres, wheat fibre, oat fibre, corn fibre, potato fibres, apple fibres, citrus fibres, bamboo fibres, extracted sugar beet fibres, oat fibres, soy fibres, cone fibres, beet fibres, and combinations thereof; and dietary fibres soluble under physiological conditions, such as: β-glucans, inulin, in particular native inulin, highly soluble inulin, granulated inulin, high-performance inulin, pectins, seaweed extracts including alginates, agar, carrageenan, and furcellan, gum arabic, guar gum, locust bean gum, xanthan gum, raffinose, xylose, polydextrose, lactulose, gum ghatti, traganth (tragacanth), karaya, mucillages such as psyllium, methyl cellulose (MC), and combinations thereof; as well as fibres comprising both soluble and insoluble components such as pea fibres.

It is well known to the person skilled in the art that dietary fibres occur naturally in various food sources.

Sources of dietary fibres include, but are not limited to, grains such as oats, rye, barley, such as wheat, rye, chia, barley, legumes such as peas and beans such as soybeans or lupins, fruits and vegetables apples, oranges, pears, bananas, berries, tomatoes, green beans, broccoli, cauliflower, carrots, potatoes, celery, as well as plant foods such as bran, nuts, and seeds (such as flax seeds) or indigestible animal products such as chitins. In addition to these naturally occurring dietary fibers, biosynthetic or fermentation products such as dextran, xanthan and curdan can be used as well as chemically modified materials such as carboxymethyl cellulose (CMC), methylcellulose, hydroxypropylcellulose, hydroxypropylmethyl cellulose, hydroxyethyl cellulose (HEC), low methoxy pectin and propylene glycol alginate, or synthetic fibres such as polyvinylpyrrolidone, carboxyvinyl polymers and polyethylene oxide polymers.

Such compounds are well known to those skilled in the art. As used herein, at least one dietary fibre may be used or a combination of two or more dietary fibres. In addition, also combinations of one or more soluble and one or more insoluble dietary fibre can be advantageously used for the purpose of the present invention. Preferably, the dietary fibre(s) used within the context of the present invention is an/are authorized food additive(s). Even more preferred, the dietary fibre(s) used within the context of the present invention is a/are naturally occurring dietary fibre(s), and preferably bioderived.

### Combinations of Fireproofing Agents as Specified Herein:

The powdered composition according to the first aspect of the present invention comprises at least one fireproofing agent specified above, meaning that mixtures of two or more different fireproofing agents are likewise suitable for the purpose of the present invention.

According to a further preferred embodiment the present invention relates to a powdered composition according to the first aspect of the invention, wherein the fireproofing agent is selected from the group consisting of sodium chloride, calcium chloride, magnesium stearate, polysaccharides, sugar alcohols, proteins, water-soluble and/or water-insoluble dietary fibres, plant-based oils and fats, plant extracts, or mixtures thereof, as specified herein.

In a further preferred variant of the present invention, the powdered composition comprises or consists of from 1% to 80% by weight, and preferably from 5% to 60% by weight, and even more preferred from 10% to 40% by weight, of the fireproofing agent as specified herein based on the total weight of the powdered composition.

If the fireproofing agent(s) according to the present invention is/are comprised within said ranges, considerable improvements in the minimum ignition energy (MIE) and the burning rates of the powdered compositions could be achieved without negatively influencing the compositions properties such as its odour, colour or stability.

Mixtures of two or more fireproofing agents can also be advantageously employed within the context of the present invention. Suitable and/or referred fireproofing agents of said combinations or mixtures of fireproofing agents are specified above.

Thus, preferably, according to another preferred variant of the powdered composition according to the invention, the fireproofing agent comprises or consists of:
a mixture of at least one mineral salt with at least one salt of an organic acid,
a mixture of at least one polysaccharide, at least one sugar alcohol, at least one protein, at least one water-soluble and/or water-insoluble dietary fibre, at least one plant extract with at least one salt of an organic acid, or
a mixture of at least one polysaccharide, at least one sugar alcohol, at least one protein, at least one water-soluble and/or water-insoluble dietary fibre, at least one plant extract with at least one mineral salt.

If the fireproofing agent comprises or consists of a mixture as specified above, the at least polysaccharide, the at least one sugar alcohol, the at least one protein, the at least one water-soluble and/or water-insoluble dietary fibre, the at least one plant extract and the at least one mineral salt can be any of the substances specified above. However, the at least one salt of an organic acid can be any the salt of any organic acid, irrespective of its chain length. Preferably however the organic acid has a chain length of 13 or more carbon atoms.

The salts of organic acids according to the present invention suitable for the combination with at least polysaccharide, at least one sugar alcohol, at least one protein, at least one water-soluble and/or water-insoluble dietary fibre, at least one plant extract or at least one mineral salt, include the salts of barium, lithium, sodium, zinc, bismuth, chromium, cobalt, copper, potassium, strontium, titanium, tungsten, magnesium, caesium, iron, nickel, silver, aluminium, tin, or calcium of organic acids, and in particular the corresponding salts of saturated and/or unsaturated organic acids as well as derivatives thereof.

Preferably, according to the present invention the fireproofing agent is the salt of a saturated and/or unsaturated (monounsaturated or polyunsaturated) organic acid having a chain length of 13 or more carbon atoms combined with at least polysaccharide, at least one sugar alcohol, at least one protein, at least one water-soluble and/or water-insoluble dietary fibre, at least one plant extract or at least one mineral salt.

According to another particularly preferred embodiment of to the present invention the fireproofing agent is the salt of a saturated and/or unsaturated (monounsaturated or polyunsaturated) organic acid having a chain length of 1 to 12 carbon atoms combined with at least polysaccharide, at least one sugar alcohol, at least one protein, at least one water-soluble and/or water-insoluble dietary fibre, at least one plant extract or at least one mineral salt.

Surprisingly, it was found that such combinations advantageously improve the minimum ignition energy (MIE) and the burning rates of powdered compositions. In particular, it was found that only local burning occurred and/or that no fire propagation took place indicating a synergistical effect based on the combination of two or more different fireproofing agents.

According to a further preferred variant of the powdered composition according to the invention, the fireproofing agent comprises or consists of:
a mixture of a mineral salt with a salt of an organic acid,
a mixture of a polysaccharide, a sugar alcohol, a protein, a water-soluble and/or water-insoluble dietary fibre, a plant extract with a salt of an organic acid, or
a mixture of a polysaccharide, a sugar alcohol, a protein, a water-soluble and/or water-insoluble dietary fibre, a plant extract with a mineral salt.

The powdered composition comprises or consists of from 1% to 80% by weight, and preferably from 5% to 60% by weight, and even more preferred from 10% to 40% by weight, of the combined fireproofing agents, i.e. of the mixture of two or more fireproofing agents, based on the total weight of the powdered composition.

### Powdered Composition:

The powdered composition according to the first aspect of the present invention moreover comprises or consists of either a spray-drying product (variant 1), or alternatively, a composition comprising or consisting of at least one microcapsule (variant 2), and optionally a carrier system, in addition to the effective amount of a fireproofing agent specified above.

According to an alternative variant, the powdered composition according to the present invention comprises a combination of a spray-drying product as well as microcapsules.

It was surprisingly found that the powdered compositions according to the invention are secure although exhibiting a low density.

It was also surprisingly found that the powdered compositions according to the invention are secure although exhibiting a low water content.

Furthermore, it was surprisingly found that the powdered compositions according to the invention are secure although exhibiting small particle sizes.

These improved safety properties are based on the addition of substances as specified herein acting as fireproofing agents.

The substances as specified herein and as set out in the claims share a common property as they can suitably be used as fireproofing agents. Based on the use/incorporation of an effective amount of these substances as additives, it is possible to achieve burning rates of ≤2.22 mm/s for various powdered formulations and a significant improvement of the MIE. In addition, it was surprisingly found that said fireproofing agents are harmless to health, environmentally friendly and also neutral in taste and can advantageously improve the burning rates of powdered compositions. In particular, it was found that only local burning occurred and/or that no fire propagation took place indicating a synergistical effect based on the combination of two or more different fireproofing agents. These common properties of the substances specified herein were previously not known.

The term “fireproofing agent” is merely used to denote the substances in their effect within the scope of the present invention and in view of powdered compositions rather than defining those substances as such.

Preferred is a powdered composition comprising or consisting of either a spray-drying product, or alternatively, a composition comprising or consisting of at least one microcapsule, and optionally a carrier system, further comprising or consisting of an effective amount of a fireproofing agent selected from the group consisting of polysaccharides, sugar alcohols, proteins, water-soluble and/or water-insoluble dietary fibres, plant-based oils and fats, salts of fatty acids having a chain length of 13 or more carbon atoms such as magnesium stearate, plant extracts such as tea extracts, or mixtures thereof.

Moreover, excellent results were achieved for fine powders exhibiting particles sizes D50 of 50 μm or less. The particle size distribution D50 denotes that 50% of the particle sizes in the sample are smaller and 50% larger than the corresponding value. Such particles are particularly prone to burning.

Thus, in a further preferred variant, the particle size D50 of the powdered composition is 50 μm or less, preferably 30 μm or less and even more preferred 20 μm or less.

Depending on the type of the powdered composition the powdered composition can comprise a composition comprising or consisting of at least one microcapsule and optionally a carrier system (variant 2). Such powdered compositions can be prepared by e.g. (spray-) drying an aqueous dispersion comprising at least one microcapsule which preferably encapsulates an active ingredient (such as a flavour or a fragrance or flavour mixture or fragrance mixture) in its core. Thereby, the microcapsules comprise a shell material selected from amongst others polyurea-based shell materials, polyurethane-based shell materials, polyolefin-based shell materials, polysaccharide-based shell materials, polyamine reacted with polyisocyanates, melamine-formaldehyde-based shell materials and the like.

Furthermore, the microcapsule(s) can comprise a carrier for the microcapsule(s) such as any water-soluble acid, organic or inorganic acids, and salts and conjugated bases thereof, including alkali metals, alkaline earth metals, and transition metals and complexes thereof. Preferably the solid carrier can be any particles, preferably porous particles, suitable to vehicle the active ingredients/microcapsules. Advantageous carriers which may be mentioned are (preferably also spray-dried) silicon dioxide (silica, silica gel), carbohydrates and/or carbohydrate polymers (polysaccharides), cyclodextrins, starches, degraded starches (starch hydrolysates), chemically or physically modified starches, modified celluloses, gum arabic, ghatti gum, tragacanth, karaya gum, carrageenan, guar gum, locust bean gum, alginates, pectin, inulin or xanthan gum. Preferred starch hydrolysates are maltodextrins and dextrins. Preferred carriers are silicon dioxide, gum arabic and maltodextrins, wherein maltodextrins having DE values in the range of from 5 to 20 are in turn preferred. It is not important which plant originally produced the starch for producing the starch hydrolysates. Corn-based starches as well as starches from tapioca, rice, wheat or potatoes are suitable and readily available. The carriers can thereby also act as flow aids, such as, for example, silicon dioxide. Preferred solid carrier materials, which may be a component of a preparation according to the invention are hydrocolloids, such as starches, degraded starches, chemically or physically modified starches, dextrins, (powdery) maltodextrins (preferably with a dextrose equivalent value of 5 to 25, preferably of 10-20), lactose, silicon dioxide, glucose, modified celluloses, gum arabic, ghatti gum, traganth, karaya, carrageenan, pullulan, curdlan, xanthan gum, gellan gum, guar flour, carob bean flour, alginates, agar, pectin and inulin and mixtures of two or more of these solids, in particular maltodextrins (preferably with a dextrose equivalent value of 15-20), lactose, silicon dioxide and/or glucose. Such carriers can further be porous inorganic materials such as light sulfate, silica gels, zeolites, gypsum, clays, clay granules, aerated concrete, etc., or organic materials such as woods and cellulosic based materials.

According to an alternative and even more preferred variant, the powdered composition comprises or consists of a spray-drying product, preferably enclosing an active ingredient such as flavour or fragrance in its structure/matrix (variant 1). Thereby, the active ingredient is freely dispersed, i.e. not encapsulated in core-shell microcapsules, within the continuous (polymeric) matrix of the spray-drying product/particle.

The spray-drying product can additionally comprise a carrier material as specified above.

This means that the powdered composition can comprise or consist of particles preferably comprising an active ingredient or composition (such as fragrance substances or flavour substances and mixtures of different fragrance substances or flavour substances) which either are
encapsulates in form of core-shell microcapsules and/or which are
freely dispersed and thus not encapsulated in core-shell microcapsules within the continuous (polymeric) matrix of a spray-drying product/particle,
   wherein the powdered composition further comprises at least one fireproofing agent as specified herein.

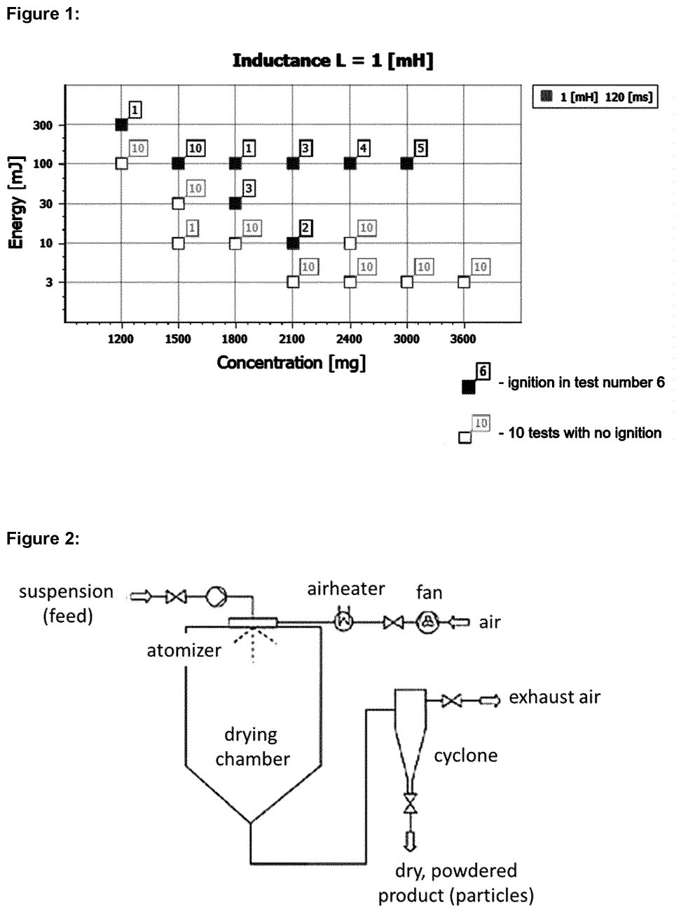

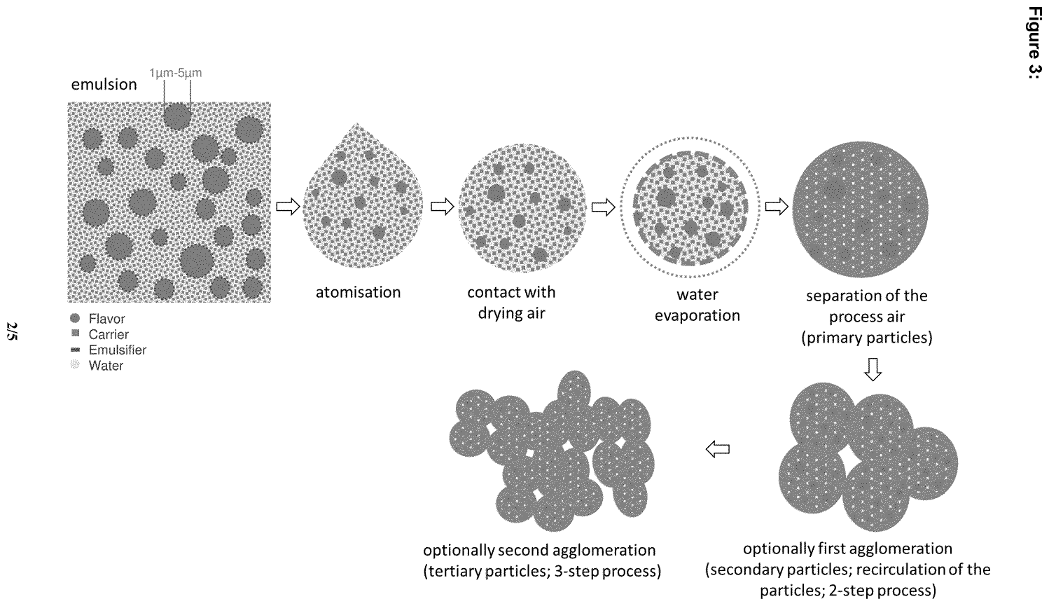

The spray-dried product or composition can be prepared, for example, by spray-drying an emulsion or dispersion optionally containing an active. FIG. 2 is a schematic representation of the exemplary preparation of powdered compositions by spray-drying, while FIG. 3 schematically visualizes the formation of the particles, i.e. the spray-drying product as such based on a one-step spray-drying process and a multi-step spray-drying process, respectively.

Such spray-dried products or compositions containing a granulated powder comprising spray-dried particles are for example obtained by a process comprising the following steps:
(i) Preparing a water phase comprising a water-soluble polymer and optionally a carrier (suspension or feed), wherein the water phase optionally comprises an emulsifier;
(ii) Optionally, preparing an oil phase optionally comprising an active ingredient such as a perfume or a flavour and optionally additional emulsifiers;
   and mixing the oil phase with the water phase to obtain an emulsion;
(iii) Spray-drying the water phase of step (i) or the emulsion of step (ii) to obtain a spray-dried product/particle.

The products of such processes, i.e. the spray-drying products, are dry, somewhat porous powders containing roughly spherical, convoluted particles with the coating polymeric material in the solid state and e.g. with the fragrance or flavour oil either dispersed as minute droplets throughout the particle, or dissolved in a solid matrix, or both, depending on the compatibility of the oil and coating material. Thereby, a very fine, nano-scaled dispersion of the water insoluble material through the water-soluble carrier material is formed. Usually, the resulting dry materials are not present as encapsulates (i.e. not encapsulated in discrete core-shell microcapsules) as discrete bodies of the water-immiscible materials are rather not formed in the dry product. The spray-drying technique preferably used herein to produce the preferred particles (i.e. the spray-drying product) according to the present invention aerosolizes droplets of said mixture (usually an emulsion) in a heated environment which allows most of the water to evaporate within a drying chamber, resulting in a virtually homogenous particle size distribution.

According to a further preferred embodiment the present invention relates to a powdered composition comprising or consisting of
a spray-drying product, and/or
a composition comprising or consisting of at least one microcapsule,
and optionally a carrier system, further comprising or consisting of
an effective amount of at least one fireproofing agent selected from the group consisting of: polysaccharides, sugar alcohols, proteins, water-soluble and/or water-insoluble dietary fibres, plant-based oils and fats, edible mineral salts such as sodium chloride or calcium chloride, salts of organic acids such as magnesium stearate, plant extracts such as tea extracts, or mixtures thereof,
   wherein the powdered composition comprises or consists of from 1% to 80% by weight, and preferably from 5% to 60% by weight, and even more preferred from 10% to 40% by weight, and most preferred 10% by weight of the at least one fireproofing agent based on the total weight of the powdered composition, and
   preferably wherein the powdered composition comprises or consists of particles preferably comprising one or more active ingredients which either are encapsulated in the form of discrete core-shell microcapsules and/or which are freely dispersed and thus not encapsulated in core-shell microcapsules within the (polymeric) matrix of the spray-drying product.

Preferably, the powdered composition according to the first aspect comprises at least one perfuming and/or flavouring active ingredient or a mixture of two or more perfuming and/or flavouring active ingredients, respectively. However, according to another variant of the present invention the substances specified herein as fireproofing agents are also considered as suitable active ingredients, which, for example, can be (additionally) incorporated into the core of the microparticles according to the present invention. Thereby, the active ingredients can be selected independently of the fireproofing agent(s).

According to an embodiment of the present invention the powdered composition comprises or consists of:
a composition comprising or consisting of at least one microcapsule and optionally a carrier system, further comprising or consisting of
an effective amount of at least one fireproofing agent selected from the group consisting of: polysaccharides, sugar alcohols, proteins, water-soluble and/or water-insoluble dietary fibres, plant-based oils and fats, edible mineral salts such as sodium chloride or calcium chloride, salts of organic acids such as magnesium stearate, plant extracts such as tea extracts, or mixtures thereof,
   preferably, wherein the powdered composition comprises or consists of from 1% to 80% by weight, and preferably from 5% to 60% by weight, and even more preferred from 10% to 40% by weight, and most preferred 10% by weight of the at least one fireproofing agent based on the total weight of the powdered composition, and preferably, wherein the powdered composition comprises or consists of at least one microcapsule, and wherein one or more (preferably hydrophobic) active ingredients such as fragrances or flavours are encapsulated in said microcapsules.

According to a further preferred embodiment the powdered composition comprises or consists of:
spray-drying product, and optionally a carrier system, and
an effective amount of at least one fireproofing agent selected from the group consisting of: polysaccharides, sugar alcohols, proteins, water-soluble and/or water-insoluble dietary fibres, plant-based oils and fats, edible mineral salts such as sodium chloride or calcium chloride, salts of organic acids such as magnesium stearate, plant extracts such as tea extracts, or mixtures thereof,
   preferably, wherein the powdered composition comprises or consists of from 1% to 80% by weight, and preferably from 5% to 60% by weight, and even more preferred from 10% to 40% by weight, and most preferred 10% by weight of the at least one fireproofing agent based on the total weight of the powdered composition, and preferably, wherein the powdered composition comprises or consists of particles preferably comprising one or more (preferably hydrophobic) active ingredients such as fragrances or flavours which are freely dispersed and thus not encapsulated in the form of core-shell microcapsules within the (polymeric) matrix of the spray-drying product.

Suitable perfuming active ingredients are for example:

extracts of natural raw materials and also fractions thereof or components isolated therefrom; single fragrance substances from a group of hydrocarbons; aliphatic alcohols; aliphatic aldehydes and acetals; aliphatic ketones and oximes; aliphatic sulfur-containing compounds; aliphatic nitriles; esters of aliphatic carboxylic acids; formates, acetates, propionates, isobutyrates, butyrates, isovalerates, pentanoates, hexanoates, crotonates, tiglinates and 3-methyl-2-butenoates of acyclic terpene alcohols; acyclic terpene aldehydes and ketones as well as their dimethyl and diethyl acetals; formates, acetates, propionates, isobutyrates, butyrates, isovalerates, pentanoates, hexanoates, crotonates, tiglinates and 3-methyl-2-butenoates of cyclic terpene alcohols; cyclic terpene aldehydes and ketones; cyclic alcohols; cyclic and cycloaliphatic ethers; cyclic and macrocyclic ketones; cycloaliphatic aldehydes; cycloaliphatic ketones; esters of cyclic alcohols; esters of cycloaliphatic carboxylic acids; aromatic hydrocarbons; araliphatic alcohols; esters of araliphatic alcohols and aliphatic carboxylic acids; araliphatic ethers; aromatic and araliphatic aldehydes; aromatic and araliphatic ketones; aromatic and araliphatic carboxylic acids and their esters; nitrogen-containing aromatic compounds; phenyl ethers and phenyl esters; heterocyclic compounds; lactones; and mixtures of the above active ingredients.

Specific examples of the abovementioned substances are known to the person skilled in the art. Fragrant substances or odorant substances and mixtures of two or more of said substances are chemical substances or compositions which are used to confer, impart and/or modulate a specific odour or odour impression. Preferably, said odour (impression) is considered as being pleasant.

The at least one flavouring or aromatic substance or flavour according to the invention (flavouring active ingredients) may also be a mixture of at least two flavouring and/or aromatic substances. Preferably it is a mixture of at least three, four or more than five flavouring substances and/or aromas. In most cases, aroma mixtures are a mixture of many aroma substances and/or aromas.

Suitable flavouring substances for the production of the powdered composition according to the present invention can be found, for example, in Steffen Arctander, “Riechstoffe” in “Perfume and Flavor Chemicals”, self-publication, Montclair, N. J. 1969; H. Surburg, J. Panten, in “Common Fragrance and Flavor Materials”, 6th Edition, Wiley-VCH, Weinheim 2016, for example: esters (saturated or unsaturated) such as ethyl butyrate, allyl capronate, benzyl acetate, methyl salicylate; organic acids (saturated and unsaturated) such as butyric acid, acetic acid, methyl butyric acid, caproic acid; alcohols (saturated and unsaturated) such as e.g. ethanol, propylene glycol, octenol, cis-3-hexanol, benzyl alcohol, phenylethyl alcohol; aldehydes (saturated and unsaturated) such as acetaldehyde, isobutyraldehyde, nonadienal, benzaldehyde, 3-phenylacetaldehyde; ketones such as menthone; ethers such as 4-hydroxy-5-methylfuranone, 3-hydroxy-4,5-dimethyl-2-(5H)-furanone, 2,5-dimethyl-3-hydroxy-2 (3H)-furanone, 2 (5)-ethyl-4-hydroxy-5 (2)-methyl-3 (2H)-furanone, p-methoxybenzaldehyde, guaiacol, methoxyvinylphenol; acetals such as Acetaldehyde diethyl acetal; lactones such as gamma-decalactone; terpenes such as limonene, linalool, terpinene, terpineol, citral (geranial and neral), menthol; sulfides and disulfides such as dimethyl sulfide, difurfuryl disulfide, methylthiopropanal; thiols such as e.g. methylfuranthiol; pyrazines and pyrrolines such as methylpyrazine, acetylpyrazine, 2-propionylpyrroline, 2-acetylpyrroline, acetophenone, allyl capronate, alpha-ionone, beta-ionone, anisaldehyde, anisyl acetate, anisyl formate, benzaldehyde, benzothiazole, benzyl acetate, benzyl alcohol, Benzyl benzoate, beta-ionone, butyl butyrate, butyl capronate, butylidene phthalide, carvone, camphene, caryophyllene, cineole, cinnamyl acetate, citral, citronellol, citronellal, citronellyl acetate, cyclohexyl acetate, cymene, damascone, decalactone, dihydrocoumarin, dimethylanthranilate, dimethylanthranilate, dodecalactone, ethoxyethyl acetate, ethyl butyric acid, ethyl butyrate, ethyl caprinate, ethyl capronate, ethyl crotonate, ethyl furaneol, ethyl guaiacol, ethyl isobutyrate, ethyl isovalerianate, ethyl lactate, ethyl methyl butyrate, ethyl propionate, eucalyptol, eugenol, ethyl heptylate, 4-(p-hydroxyphenyl)-2-butanone, gamma-decalactone, geraniol, geranyl acetate, geranyl acetate, grapefruitaldehyde, methyl dihydrojasmonate (e.g. Hedion®), heliotropin, 2-heptanone, 3-heptanone, 4-heptanone, trans-2-heptenal, cis-4-heptenal, trans-2-hexenal, cis-3-hexenol, trans-2-hexenoic acid, trans-3-hexenoic acid, cis-2-hexenyl acetate, cis-3-hexenyl acetate, cis-3-hexenyl capronate, trans-2-hexenyl capronate, cis-3-hexenyl formate, cis-2-hexyl acetate, cis-3-hexyl acetate, trans-2-hexyl acetate, cis-3-hexyl formate, para-hydroxybenzylacetone, isoamyl alcohol, isoamyl isovalerianate, isobutyl butyrate, isobutyraldehyde, isoeugenol methyl ether, isopropylmethylthiazole, lauric acid, leavulinic acid, linalool, linalool oxide, linalyl acetate, menthol, menthofuran, methyl anthranilate, methyl butanol, methyl butyric acid, 2-methyl butyl acetate, methyl capronate, methyl cinnamate, 5-methylfurfural, 3,2,2-methylcyclopentenolone, 6,5,2-methylheptenone, methyl dihydrojasmonate, methyl jasmonate, 2-methylmethylbutyrate, 2-methyl-2-pentenolic acid, methylthiobutyrate, 3,1-methylthiohexanol, 3-methylthiohexyl acetate, nerol, nerylacetate, trans, trans-2,4-nonadienal, 2,4-nonadienol, 2,6-nonadienol, 2,4-nonadienol, nootkatone, delta octalactone, gamma octalactone, 2-octanol, 3-octanol, 1,3-octenol, 1-octyl acetate, 3-octyl acetate, palmitic acid, paraldehyde, phellandrene, pentandione, phenylethyl acetate, phenylethyl alcohol, phenylethyl alcohol, phenylethyl isovalerianate, piperonal, propionaldehyde, propyl butyrate, pulegone, pulegol, sinensal, sulfurol, terpinols, 8,3-thiomenthanone, 4,4,2-thiomethylpentanone, thymol, delta-undecalactone, gamma-undecalactone, valencene, valeric acid, vanillin, acetoin, ethylvanillin, ethylvanillin isobutyrate (=3-ethoxy-4-isobutyryloxybenzaldehyde), 2,5-Dimethyl-4-hydroxy-3 (2H)-furanone and its derivatives (preferably homofuraneol (=2-ethyl-4-hydroxy-5-methyl-3 (2H)-furanone), homofuronol (=2-ethyl-5-methyl-4-hydroxy-3 (2H)-furanone and 5-ethyl-2-methyl-4-hydroxy-3 (2H)-furanone), maltol and maltol derivatives (preferably ethylmaltol), coumarin and coumarin derivatives, gamma-lactones (preferably gamma-undecalactone, gamma-nonalactone, gamma-decalactone), delta-lactones (preferably 4-methyl-deltadecalactone), massoilactone, delta-decalactone, tuberolactone), methylsorbate, divanillin, 4-hydroxy-2 (or 5)-ethyl-5 (or 2)-methyl-3 (2H) furanone, 2-hydroxy-3-methyl-2-cyclopentenone, 3-hydroxy-4,5-dimethyl-2 (5H)-furanone, isoamyl acetate, ethyl butyrate, n-butyl butyrate, isoamyl butyrate, ethyl 3-methyl butyrate, ethyl n-hexanoate, allyl n-hexanoate, n-butyl n-hexanoate, ethyl n-octanoate, ethyl 3-methyl-3-phenylglycidate, ethyl 2-trans-4-cis-decadienoate, 4-(p-hydroxyphenyl)-2-butanone, 1,1-dimethoxy-2,2,5-trimethyl-4-hexane, 2,6-dimethyl-5-hepten-1-al and phenylacetaldehyde, 2-methyl-3-(methylthio) furan 2-methyl-3-furanthiol, bis(2-methyl-3-furyl) disulfide, furfuryl mercaptan, methional, 2-acetyl-2-thiazoline, 3-mercapto-2-pentanone, 2,5-dimethyl-3-furanthiol, 2,4,5-trimethylthiazole, 2-acetylthiazole, 2,4-dimethyl-5-ethylthiazole, 2-acetyl-1-pyrroline, 2-methyl-3-ethylpyrazine, 2-ethyl-3,5-dimethylpyrazine, 2-ethyl-3,6-dimethylpyrazine, 2,3-diethyl-5-methylpyrazine, 3-isopropyl-2-methoxypyrazine, 3-isobutyl-2-methoxypyrazine, 2-acetylpyrazine, 2-pentylpyridine, (E,E)-2,4-decadienal, (E,E)-2,4-nonadienal, (E)-2-octenal, (E)-2-nonenal, 2-undecenal, 12-methyltridecanal, 1-penten-3-one, 4-hydroxy-2,5-dimethyl-3 (2H)-furanone, guaiacol, 3-hydroxy-4,5-dimethyl-2 (5H)-furanone, 3-Hydroxy-4-methyl-5-ethyl-2 (5H)-furanone, cinnamic aldehyde, cinnamic alcohol, methyl salicylate, isopulegol and (here not explicitly mentioned) stereoisomers, enantiomers, position isomers, diastereomers, cis/trans isomers or epimers of these substances.

The minimum ignition energy (MIE) describes the lowest energy that is sufficient in a discharge to ignite a substance or composition and is thus a safety-related parameter for assessing the ignitability of explosive atmospheres such as electrostatic sparks. Up to 10 mJ, a sample can already be ignited by a slight discharge of static energy. This energy can occur during any processing step, even if the equipment is earthed according to regulations. Above 10 mJ, the MIE is considered manageable because proper grounding is sufficient to prevent discharges greater than 10 mJ. Therefore, a high MIE greater than 10 mJ should be aimed for in any case.

The addition of an effective amount of the fireproofing agents according to the present invention advantageously reduces the hazardousness oft the corresponding powdered product formulations by considerably and efficiently improving the MIE-values of the powdered compositions corresponding to low reactivities and thus indicating increased resistances to external ignition although exhibiting extremely low particle sizes. Said additives as specified herein, prevent the dust explosion risk when said powdered compositions are manufactured, handled, transported, processed, or incorporated into a consumer product.

Preferably, the at least one fireproofing agent as specified herein is comprised in an amount of from 1% to 80% by weight, and preferably from 5% to 60% by weight, further preferred from 10% to 40% by weight, even more preferred from 10% to 20% by weight, and most preferred in an amount of 10% by weight. If the agent is comprised in these amount excellent fireproofing properties could be achieved when incorporated in powdered formulations.

Based thereon, the powdered composition according to the invention has a minimum ignition energy of at least 10 mJ, preferably of at least 30 mJ, more preferred of at least 100 mJ and most preferred of at least 300 mJ.

The burning rate is measured as the burning time of a 10 cm long sample. Generally, 10 cm of the piled-up powder should burn for longer than 45 seconds. Faster burning indicates the tested sample as highly flammable, which means that special precautions must be taken for transport and the like causing higher costs and additional safety precautions.

It was surprisingly found that all samples resulted in an improved burning rate, whereby the critical value could be reached for some substances such as for CaCl₂) or pea fibre as fireproofing additives. Here, only local burning or brief burning occurred, but not the entire powder section burned. Fibres and salts show particularly good results here, which could certainly be controlled via the concentration.

Surprisingly, it was found that the fireproofing agents according to the present invention and their combinations advantageously improve the burning rates of powdered compositions. In particular, it was found that only local burning occurred and/or that no fire propagation took place indicating a synergistical effect based on the combination of two or more different fireproofing agents.

In this view, the powdered composition according to the present invention preferably has a burning rate of ≤4 mm/s, more preferred of ≤3 mm/s, further preferred of ≤2.2 mm/s, and most preferred of ≤1.8 mm/s.

According to a particularly preferred variant of the present invention the powdered composition has a burning rate of ≤2.2 mm/s.

The minimum ignition temperature (MIT) is a further critical parameter for the safety of powdered compositions and is defined as the lowest temperature of a hot surface, determined under prescribed test conditions, at which the most ignitable air/dust mixture ignites. Surprisingly it was found that all fireproofing agents according to the present invention allow for the improvement (i.e. increase) of the minimum ignition temperature for whirled-up dust clouds, i.e. dust/air mixtures and thus for the preparation of more stable, i.e. less dangerous powdered compositions. The minimum ignition temperature (MIT) is a particularly relevant parameter in the evaluation of the probability of ignition by hot surfaces, such as those caused by moving parts being heated by friction during transport.

Therefore, preferably, the powdered composition according to the present invention has a minimum ignition temperature of a whirled-up dust cloud of ≥300° C., preferably of ≥350° C., more preferred of ≥400° C. and most preferred of ≥420° C.

Based on the determined safety parameters such as the minimum ignition energy (MIE) and the minimum ignition temperatures (MIT) of dust layers or clouds comprising the fireproofing agents according to the present invention or mixtures thereof, it can be concluded that the fireproofing agents according to the present invention are highly efficient without negatively affecting the product properties such at the taste of the powdered composition.

In addition, it was surprisingly found that although the additives make the powders more flowable and dusty (which would actually speak for an increased ignitability), the fireproofing additives as specified herein are excellent in advantageously modifying the burning rate and the MIE of various powdered formulations.

It was further surprisingly found that the addition of polysaccharides and/or dietary fibres such as plant gums and other high molecular weight substances show a high energy consumption during ignition (strong endothermic transition). Thus, these substances and mixtures thereof are particularly preferred as the fireproofing component in the compositions of the present invention.

Accordingly, in a further preferred variant the at least one fireproofing agent effect is preferably selected from the group consisting of polysaccharides, sugar alcohols, proteins, water-soluble and/or water-insoluble dietary fibres, or mixtures thereof, and as specified herein.

According to a second aspect the present invention relates to a method for the preparation of a powdered composition, comprising the following steps:
(a) providing an aqueous dispersion, optionally comprising microcapsules, comprising a fireproofing agent selected from the group consisting of polysaccharides, sugar alcohols, proteins, water-soluble and/or water-insoluble dietary fibres, plant-based oils or fats, edible mineral salts such as sodium chloride or calcium chloride, salts of organic acids such as magnesium stearate, plant extracts such as tea extracts, or mixtures thereof, and optionally a carrier material, and
(b) spray-drying the dispersion of step (a) to obtain a powdered composition.

Spray-drying is a method of preparing a dry powder with homogeneous and fine particle sizes from a liquid or slurry by drying with a hot gas as drying medium. Spray-drying can be used for the formation of matrix microparticles or powdered particles entrapping the active(s) based on the inclusion of active ingredients or compositions in a matrix in terms of particles of solid dispersions, depending on the composition and conditions. Alternatively, spray-draying can be used for drying microparticle dispersions.

This means the product of step (b) can be defined as a microparticle preferably comprising a (hydrophobic) active ingredient or composition such as fragrances or flavours which either can be encapsulates in form of core-shell microcapsules (variant 2) and/or freely dispersed and thus not encapsulated in core-shell microcapsules within the continuous (polymeric) matrix of the resulting spray-drying product/particle (variant 1).

According to one variant, said aqueous dispersion of step (a) can comprises at least one microcapsule as well as optionally a carrier material. The at least one microcapsule of the dispersion which preferably encapsulates an active ingredient (such as a flavour or a fragrance or a flavour mixture or a fragrance mixture) in its core preferably comprises a shell material selected from amongst others polyurea-based shell materials, polyurethane-based shell materials, polyolefin-based shell materials, polysaccharide-based shell materials, polyamine reacted with polyisocyanates, melamine-formaldehyde-based shell materials and the like.

Additionally, the aqueous dispersion comprises at least one fireproofing agent according to the invention. Upon spray-drying the water is evaporated, giving discrete microparticles comprising the fireproofing agent(s) according to the present invention. Thereby, the fireproofing agent is deposited onto the discrete microcapsules, i.e. microcapsules are coated with the fireproofing agent according to the present invention.

Alternatively, the dry/solid fireproofing agent is blended with a spray-dried powder comprising the active ingredient(s) encapsulated in the polymeric shell or carrier material, i.e. the fireproofing agent is blended with the as-prepared core-shell microcapsules comprising the active ingredient(s) as core materials.

According to a preferred embodiment the present invention thus relates to a method for the preparation of a powdered composition, wherein the aqueous dispersion of step (a) comprises at least one microcapsule, wherein the at least one microcapsule preferably comprises the active ingredient(s). Additionally, the aqueous dispersion of step (a) optionally comprises a carrier system. Some of the carrier system or all of it can be substituted with the fireproofing agent(s).

However, according to an even more preferred variant the powdered composition is a spray-drying product preferably not containing discrete core-shell microcapsules. Thus, in a preferred variant of the second aspect the present invention relates to a method for the preparation of a powdered composition, wherein the product of step (b) is a spray-drying product, and preferably a spray-dried product not containing discrete microcapsules.

Again, the at least one fireproofing agent is either incorporated into the carrier material/the matrix of the spray-drying product or alternatively the spray-drying product is admixed (dry blend) with the at least one fireproofing agent and the particles of the spray-drying product are coated with the at least one fireproofing agent.

Preferably, the at least one fireproofing agent is added to the aqueous dispersion (in step (a)) and subsequently spray-dried resulting in the incorporation of the at least one fireproofing agent into the solidified carrier matrix.

The final particles of the spray-drying product are particles preferably comprising at least one active ingredient such as a fragrance or a flavour freely dispersed in the surrounding (carrier) matrix.

According to a preferred variant the active ingredient(s) thus is/are included in a carrier matrix in form of particles of solid dispersions, the carrier matrix forming the continuous phase of the solid dispersion. Thus preferably, the aqueous dispersion of step (a) preferably comprises a carrier material.

Consequently, in a preferred embodiment, according to the method for the preparation of a powdered composition according to the invention, the dispersion of step (a) comprises an additional carrier.

Additionally, as shown in FIG. 3 the as-obtained primary particles (i.e. the spray-drying product or the as-obtained microcapsules) can be recirculated in order to form agglomerates (i.e. secondary or higher particles).

Thus, according to a further preferred embodiment the method for the preparation of a powdered composition further comprises the step of:
(c) agglomeration after step (b).

Based on the formation of bigger particles the safety of a powdered composition can be further increased, in particular of powdered compositions comprising volatile and flammable component such as flavours and/or fragrances.

Preferably, the powdered composition prepared according to the method of the second aspect, comprises ≤6% by weight and preferably ≤5% by weight of (remaining) water based on the total weight of the powdered composition.

The fireproofing agents according to the present invention can advantageously be incorporated into a variety of powdered compositions such as flavoured or perfumed powdered composition and especially compositions or products or product formulations comprising volatile and flammable substances/ingredients.

Consequently, the present invention further relates to the use of a fireproofing agent selected from the group consisting of polysaccharides, sugar alcohols, proteins, water-soluble and/or water-insoluble dietary fibres, plant-based oils, edible mineral salts such as sodium chloride or calcium chloride, salts of organic acids such as magnesium stearate, plant extracts such as tea extracts, or mixtures thereof for the preparation of a powdered composition as specified herein.

Based on the excellent fireproofing properties, the present invention also relates to the use of a fireproofing agent selected from the group consisting of polysaccharides, sugar alcohols, proteins, water-soluble and/or water-insoluble dietary fibres, plant-based oils, edible mineral salts such as sodium chloride or calcium chloride, salts of organic acids such as magnesium stearate, plant extracts such as tea extracts, or mixtures thereof for reducing the combustion potential of a powdered composition.

Based on the excellent fireproofing properties, the present invention preferably also relates to the use of a fireproofing agent selected from the group consisting of polysaccharides, sugar alcohols, proteins, water-soluble and/or water-insoluble dietary fibres, plant-based oils, salts of fatty acids such as magnesium stearate having a chain length of 13 or more carbon atoms, plant extracts such as tea extracts, or mixtures thereof for reducing the combustion potential of a powdered composition. It has been found that these substances can be used to particular advantage to effectively reduce the combustion potential of powdered compositions.

Such less dangerous powdered composition can be safer used as or for the preparation of consumer products due to their improved properties and the reduced risks upon storage, handling and processing. Thus, another aspect of the present invention relates to the use of powdered composition according to the present invention for the preparation of consumer products as well as to the corresponding consumer products comprising a powdered composition according to the invention.

According to a preferred embodiment said consumer products are powdered consumer products.

Powdered consumer products are for example cosmetic powders, powdered cleaning agents, powdered perfuming compositions and powdered fragrance enhancers, food products such as aroma mixtures, instant beverage mixtures, or instant food preparations.

### EXAMPLES

Hereinafter, the present invention is described in more detail and specifically with reference to the examples, which however are not intended to limit the present invention.

### Example 1: Burning Number

The burning number is a criterion for the spread of fire after local exposure to a sufficiently strong ignition source and is therefore an important parameter for the fire behaviour of solids and the fire protection concept based on it. The burning number is measured according to VDI 2263-1. Accordingly, the substance or composition is finely grounded and dried. Subsequently said dust sample is placed on a fireproof plate over a length of about 4 cm, a width of about 2 cm and a height of about 0.6 cm and ignited using a glowing platinum wire at one end of the sample.

Based on the burning behaviour of the dust sample the burning number is determined based on the following classification (see Table 1):

TABLE 1
Classification of powdered substances and compositions in terms
of their burning number based on their burning behaviour.

| | | Burning |
|---|---|---|
| Description of the burning behaviour | | number |
| No spreading | No burning | 1 |
| of fire | Short burning and rapid extinguishing | 2 |
| | Local burning or smouldering with at | 3 |
| | most slight spreading | |
| Spread of fire | Glow-through without sparks | 4 |
| | (smouldering fire) or slow, flameless | |
| | decomposition | |
| | Spreading of an open fire or burning off | 5 |
| | under sparking | |
| | Deflagration-like burning or rapid, | 6 |
| | flameless decomposition | |

Within the framework of the present invention different powdered compositions were tested. The powdered samples were prepared using an aqueous beverage formulation (“peach mix”, see Table 2) comprising different potential fireproofing agents, followed by spray-drying the aqueous dispersions to obtain dry and powdered products.

TABLE 2
Composition of the spray-dried beverage
formulation or aroma preparation (“peach
mix” or “peach flavour”).

| Ingredient | Amount [g] |
|---|---|
| Gummi Arabicum, type Senegal | 200 |
| Maltodextrin from corn DE17-20 | 500 |
| Mannitol D | 100 |
| Peach flavour | 200 |
| Water | 1025 |

TABLE 3
Burning number and burning behaviour of powdered formulations
of a peach flavour (“peach mix”, see Table 2) with
different fireproofing agents. The burning number or combustion
factor (CF) characterizes the fire and combustion behaviour
of a powdered substance or composition (Scale: 1 (no burning)
to 6 (deflagration-like burning or rapid, flameless decomposition)
measured according to VDI 2263-1).

| Peach flavour plus different | | |
|---|---|---|
| fireproofing agents (in powdered | Burning | |
| form) | number | Burning behaviour |
| Peach mix + 10 wt.-% TiO₂ | 5 | Rapid flame propagation |
| Peach mix + 20 wt.-% TiO₂ | 5 | Rapid flame propagation |
| Peach mix + 30 wt.-% TiO₂ | 5 | Rapid flame propagation |
| Peach mix + 10 wt.-% Mannitol | 5 | Rapid flame propagation |
| Peach mix + 30 wt.-% Mannitol | 5 | Rapid flame propagation |
| Peach mix + 10 wt.-% Trisodium | 5 | Rapid flame propagation |
| citrate (Na₃C₆H₅O₇) | | |
| Peach mix + 20 wt.-% Trisodium | 3 | Slow flame spread and |
| citrate (Na₃C₆H₅O₇) | | extinguishing |
| Peach mix + 30 wt.-% Trisodium | 2 | Local burning and rapid |
| citrate (Na₃C₆H₅O₇) | | extinguishment |
| Peach mix + 10 wt.-% CaCl₂ | 5 | Rapid flame propagation |
| Peach mix + 30 wt.-% CaCl₂ | 4 to 5 | Rapid flame propagation |

As can be seen from the above results only trisodium citrate shows an improvement of the burning number and consequently a considerable reduction of the spread of fire after local exposure to a sufficiently strong ignition source.

### Example 2: Burning Rate and Minimum Ignition Energy/Temperature—Preparation of a Powdered Composition Comprising a Fireproofing Agent by Spray-Drying an Aqueous Dispersion Comprising a Fireproofing Agent

In order to analyse the burning behaviour of product formulations comprising different fireproofing agents according to the present invention, an aroma formulation (“peach mix”) as specified in Table 2 was provided. Therefore, the ingredients according to Table 2 were homogenized using a high shear mixer. Subsequently, different fireproofing agents according to the present invention were added (partially replacing the maltodextrin-component of the beverage formulation). For the preparation of the different mixtures an Ultra Turray stirrer as used for 4 minutes at 10,000 rpm at room temperature. In order to improve the particle formation, the viscosity was adjusted to 120 to 130 mPas using additional water. Finally, the resulting mixtures were spray-dried to obtain dry and powdered products (180-190° C. inlet temperature; 80-90° C. outlet temperature; dryer: Mobile Minor from GEA with a max. evaporator capacity approx. 2 L/h).

The burning rates and minimum ignition energies (MIE) of said powdered compositions/bends were determined according to VDI 2263 as follows:

The burning rate is measured using a V-shaped metal channel which is 10 cm long and wherein the sample is placed, with a cross-sectional dimension of approximately 6 mm. Subsequently, the sample is ignited on one end and the corresponding time is measured until the flame front reaches to the other end of said V-shaped metal channel. Consequently, the burning rate is expressed in terms of the sample length over time at a given pressure and temperature. In the present case the burning rate was determined at ambient pressure and at room temperature and is expressed as the time in seconds which the flame front required to reach to the other end of said V-shaped metal channel. 10 cm of the piled-up powder should burn for longer than 45 seconds. Faster burning indicates the tested sample as highly flammable, which means that special precautions must be taken for transport.

The minimum ignition energy (MIE) describes the lowest energy that is sufficient in a discharge to ignite a fluidized substance or composition and is thus a safety-related parameter for assessing the ignitability of explosive atmospheres. According to DIN EN ISO/IEC 80079-20-2:2016-12, the MIE of a combustible dust/air mixture is the lowest electrical energy stored in a capacitor that, when discharged, is sufficient to trigger ignition of the most sensitive dust/air mixture, under specified test conditions. Such energy is just sufficient to ignite the most readily ignitable dust/air mixture at atmospheric pressure and room temperature while varying the parameters of the electrical discharge circuit. Thereby, the energy of 10 mJ is considered particularly critical. Up to 10 mJ, a sample can already be ignited by a slight discharge of static energy. This energy can occur during any processing step, even if the equipment is earthed according to regulations. Above 10 mJ, the MIE is considered manageable because proper grounding is sufficient to prevent discharges greater than 10 mJ. Therefore, a high MIE greater than 10 mJ should be aimed for in any case.

According to their minimum ignition energy, dusts are thus classified as follows:
MIE>10 mJ normally susceptible to ignition
10 mJ>MIE>3 mJ particularly sensitive to ignition
MIE<3 mJ extremely sensitive to ignition.

Thereby, increasing amounts of powder with decreasing energies were ignited in order to determine the lowest energy that causes the powder to ignite using a ANKO MINOR 3.1 device operated according to DIN EN ISO/IEC 80079-20-2:2016 (combustible dust test method). The corresponding measuring scheme is visualized in FIG. 1. The measurements were accomplished at temperatures of 19 to 20° C. and at a relative humidity of 29 to 31%.

An increase in the MIE by two energy levels from 10 to 100 mJ represents a significant improvement. It means for handling that the powder is no longer ignitable by low energies that cannot be controlled, such as electrostatic discharges. Salts and fibres, but also proteins (whey protein) achieve the desired effect here.

The corresponding viscosities, the particle sizes, the remaining water content as well as the MIE-values and burning rates of the as-prepared powdered blends are shown in the table below. The beverage formulation “peach mix” does not contain an additional fireproofing agent and serves as comparative example, i.e. the first column of the table shows the original recipe/composition. In the following columns, the mannitol was replaced by other components in the same proportion.

TABLE 4
Minimum ignition energy (MIE) and burning rate of a powdered peach
mix comprising the fireproofing agents according to the invention.

| | | MCC | | | | | |
|---|---|---|---|---|---|---|---|
| | Peach | 105 [g] | Glucose | | | Whey | |
| Substance | mix | (Ø particle | syrup | Mannitol | NaCl | protein | CaLac |
| [g] | [g] | size 20 μm) | [g] | [g] | [g] | [g] | [g] |
| Microcrystalline | — | 100 | — | — | — | — | — |
| cellulose (MCC), | | | | | | | |
| Vivapur 105 | | | | | | | |
| Powdered glucose | — | — | 100 | — | — | — | — |
| syrup from corn | | | | | | | |
| DE43-47 | | | | | | | |
| Gummi Arabicum, | 200 | 200 | 200 | 200 | 200 | 200 | 200 |
| type Senegal | | | | | | | |
| Maltodextrin from | 500 | 500 | 500 | 500 | 500 | 500 | 500 |
| corn DE17-20 | | | | | | | |
| Mannitol D | 100 | — | — | 100 | — | — | — |
| NaCl, table salt | — | — | — | — | 100 | — | — |
| Whey protein | — | — | — | — | — | 100 | — |
| conc. 82% | | | | | | | |
| Peach flavour | 200 | 200 | 200 | 200 | 200 | 200 | 200 |
| Water | 1025 | 1145 | 1000 | 1025 | 900 | 1245 | 925 |
| Calcium lactate | — | — | — | — | — | — | 100 |
| pentahydrate | | | | | | | |
| (CaLac) | | | | | | | |
| Sodium | — | — | — | — | — | — | — |
| carbonate, | | | | | | | |
| Na₂CO₃ | | | | | | | |
| Calcium | — | — | — | — | — | — | — |
| chloride, | | | | | | | |
| CaCl₂ | | | | | | | |
| Microcrystalline | — | — | — | — | — | — | — |
| cellulose (MCC), | | | | | | | |
| Vivapur 200 | | | | | | | |
| Pea fibre | — | — | — | — | — | — | — |
| Magnesium | — | — | — | — | — | — | — |
| stearate | | | | | | | |
| Inulin | — | — | — | — | — | — | — |
| Guar Gum | — | — | — | — | — | — | — |
| Trisodium | — | — | — | — | — | — | — |
| citrate | | | | | | | |
| Bamboo fibre | — | — | — | — | — | — | — |
| Apple pectin | — | — | — | — | — | — | — |
| Sodium hydrogen | — | — | — | — | — | — | — |
| carbonate, | | | | | | | |
| NaHCO₃ | | | | | | | |
| Soy protein | — | — | — | — | — | — | — |
| Tea extract | — | — | — | — | — | — | — |
| Total | 2025 | 2145 | 2000 | 2025 | 1900 | 2245 | 1925 |
| Viscosity [mPas] | 104 | 126 | 112 | 109 | 117 | 97 | 109 |
| D [3, 2] [μm] | 5.557 | 8.595 | 5.358 | 5.321 | 5.911 | 7.174 | 5.78 |
| d10 [μm] | 3.823 | 5.559 | 3.736 | 3.669 | 4.130 | 5.462 | 3.81 |
| d50 [μm] | 12.294 | 21.884 | 12.025 | 12.010 | 14.606 | 16.047 | 13.51 |
| d90 [μm] | 28.099 | 57.204 | 28.915 | 28.016 | 37.704 | 37.31 | 32.03 |
| Water | 3.18 | 2.64 | 2.81 | 6.82 | 3.17 | 3.24 | 5.06 |
| content [%] | | | | | | | |
| Minimum ignition | 10 | 30 | 10 | 10 | 300 | 30 | 100 |
| energy (MIE) [mJ] | | | | | | | |
| 1. Burning | 20 | 26 | 23 | 21 | 19 | 27 | 25 |
| rate [sec] | | | | | | | |
| 2. Burning | 21 | 27 | 21 | 27 | 18 | 34 | 25 |
| rate [sec] | | | | | | | |
| Burning rate, | 21 | 27 | 22 | 24 | 19 | 31 | 25 |
| mean value [sec] | | | | | | | |

TABLE 4
continuation.

| | | | | MCC | | | |
|---|---|---|---|---|---|---|---|
| | Peach | | | 200 [g] | Pea | Magnesium | |
| Substance | mix | Na₂CO₃ | CaCl₂ | (Ø particle | fibre | stearate | Inulin |
| [g] | [g] | [g] | [g] | size 190 μm) | [g] | [g] | [g] |
| Microcrystalline | — | — | — | — | — | — | — |
| cellulose (MCC), | | | | | | | |
| Vivapur 105 | | | | | | | |
| Powdered glucose | — | — | — | — | — | — | — |
| syrup from corn | | | | | | | |
| DE43-47 | | | | | | | |
| Gummi Arabicum, | 200 | 200 | 200 | 200 | 200 | 200 | 200 |
| type Senegal | | | | | | | |
| Maltodextrin from | 500 | 500 | 500 | 500 | 500 | 500 | 500 |
| corn DE17-20 | | | | | | | |
| Mannitol D | 100 | — | — | — | — | — | — |
| NaCl, table salt | — | — | — | — | — | — | — |
| Whey protein | — | — | — | — | — | — | — |
| conc. 82% | | | | | | | |
| Peach flavour | 200 | 200 | 200 | 200 | 200 | 200 | 200 |
| Water | 1025 | 1000 | 900 | 1100 | 2150 | 1050 | 1025 |
| Calcium lactate | — | — | — | — | — | — | — |
| pentahydrate | | | | | | | |
| (CaLac) | | | | | | | |
| Sodium carbonate, | — | 100 | — | — | — | — | — |
| Na₂CO₃ | | | | | | | |
| Calcium chloride, | — | — | 100 | — | — | — | — |
| CaCl₂ | | | | | | | |
| Microcrystalline | — | — | — | 100 | — | — | — |
| cellulose (MCC), | | | | | | | |
| Vivapur 200 | | | | | | | |
| Pea fibre | — | — | — | — | 100 | — | — |
| Magnesium | — | — | — | — | — | 100 | — |
| stearate | | | | | | | |
| Inulin | — | — | — | — | — | — | 100 |
| Guar Gum | — | — | — | — | — | — | — |
| Trisodium citrate | — | — | — | — | — | — | — |
| Bamboo fibre | — | — | — | — | — | — | — |
| Apple pectin | — | — | — | — | — | — | — |
| Sodium hydrogen | — | — | — | — | — | — | — |
| carbonate, NaHCO₃ | | | | | | | |
| Soy protein | — | — | — | — | — | — | — |
| Tea extract | — | — | — | — | — | — | — |
| Total | 2025 | 2000 | 1900 | 2100 | 3150 | 2050 | 2025 |
| Viscosity [mPas] | 104 | 125 | 127 | 117 | 138 | 126 | 122 |
| D [3, 2] [μm] | 5.557 | 4.75 | 6.54 | 7.28 | 6.29 | 5.18 | 5.85 |
| d10 [μm] | 3.823 | 3.14 | 4.41 | 4.72 | 4.77 | 3.39 | 4.06 |
| d50 [μm] | 12.294 | 9.82 | 14.91 | 18.39 | 13.21 | 12.46 | 13.2 |
| d90 [μm] | 28.099 | 22.42 | 37.24 | 65.39 | 39.27 | 36.2 | 31.6 |
| Water content [%] | 3.18 | 2.29 | 4.5 | 2.68 | 3.9 | 3.28 | 3.57 |
| Minimum ignition | 10 | 1000 | 100 | 100 | 100 | 30 | 100 |
| energy (MIE) [mJ] | | | | | | | |
| 1. Burning | 20 | short | short | 26 | local | 29 | 21 |
| rate [sec] | | scorching | scorching | | burning | | |
| 2. Burning | 21 | short | short | 27 | — | 30 | 19 |
| rate [sec] | | scorching | scorching | | | | |
| Burning rate, | 21 | short | short | 27 | local | 30 | 20 |
| mean value [sec] | | scorching | scorching | | burning | | |

TABLE 4
continuation.

| | | | | | | | MCC |
|---|---|---|---|---|---|---|---|
| | | | | | | MCC | (fine) + |
| | Peach | Guar | Trisodium | Bamboo | Apple | 200 [g] | Trisodium |
| Substance | mix | Gum | citrate | fibre | pectin | (Ø particle | citrate |
| [g] | [g] | [g] | [g] | [g] | [g] | size 190 μm) | [g] |
| Microcrystalline | — | — | — | — | — | 200 | 50 |
| cellulose (MCC), | | | | | | | |
| Vivapur 105 | | | | | | | |
| Powdered glucose | — | — | — | — | — | — | — |
| syrup from corn | | | | | | | |
| DE43-47 | | | | | | | |
| Gummi Arabicum, | 200 | 200 | 200 | 200 | 200 | 200 | 200 |
| type Senegal | | | | | | | |
| Maltodextrin from | 500 | 500 | 500 | 500 | 500 | 400 | 500 |
| corn DE17-20 | | | | | | | |
| Mannitol D | 100 | — | — | — | — | — | — |
| NaCl, table salt | — | — | — | — | — | — | — |
| Whey protein | — | — | — | — | | — | — |
| conc. 82% | | | | | | | |
| Peach flavour | 200 | 200 | 200 | 200 | 200 | 200 | 200 |
| Water | 1025 | 1400 | 1025 | 1150 | 1350 | — | 1100 |
| Calcium lactate | — | — | — | — | — | — | — |
| pentahydrate | | | | | | | |
| (CaLac) | | | | | | | |
| Sodium carbonate, | — | — | — | — | — | — | — |
| Na₂CO₃ | | | | | | | |
| Calcium chloride, | — | — | — | — | — | — | — |
| CaCl₂ | | | | | | | |
| Microcrystalline | — | — | — | — | — | — | — |
| cellulose (MCC), | | | | | | | |
| Vivapur 200 | | | | | | | |
| Pea fibre | — | — | — | — | — | — | — |
| Magnesium stearate | — | — | — | — | — | — | — |
| Inulin | — | — | — | — | — | — | — |
| Guar Gum | — | 100 | — | — | — | — | — |
| Trisodium citrate | — | — | 100 | — | — | — | 50 |
| Bamboo fibre | — | — | — | 100 | — | — | — |
| Apple pectin | — | — | — | — | 100 | — | — |
| Sodium hydrogen | — | — | — | — | — | — | — |
| carbonate, NaHCO₃ | | | | | | | |
| Soy protein | — | — | — | — | — | — | — |
| Tea extract | — | — | — | — | — | — | — |
| Total | 2025 | 2400 | 2025 | 2150 | 2350 | — | 2100 |
| Viscosity [mPas] | 104 | 110 | 90 | 119 | 120 | 106 | 90 |
| D [3, 2] [μm] | 5.557 | 6.39 | 5.34 | 7.65 | 5.52 | — | 6.35 |
| d10 [μm] | 3.823 | 4.64 | 3.72 | 4.96 | 3.63 | — | 4.34 |
| d50 [μm] | 12.294 | 13.68 | 12.03 | 20.21 | 13.04 | — | 15.12 |
| d90 [μm] | 28.099 | 33.72 | 29.34 | 67.17 | 29.83 | — | 42.28 |
| Water content [%] | 3.18 | 2.96 | 5.34 | 4.01 | 3.34 | — | 2.81 |
| Minimum ignition | 10 | 100 | 1000 | 30 | 300 | — | 300 |
| energy (MIE) [m]] | | | | | | | |
| 1. Burning | 20 | 22 | — | 48 | 30 | — | — |
| rate [sec] | | | | | | | |
| 2. Burning | 21 | 25 | — | 47 | 31 | — | — |
| rate [sec] | | | | | | | |
| Burning rate, | 21 | 24 | no | 48 | 31 | too big | local |
| mean value [sec] | | | burning | | | particles | burning + |
| | | | | | | | sub. |
| | | | | | | | extinguishing |

TABLE 4
continuation.

| | | | 5 wt.-% | | |
|---|---|---|---|---|---|
| | | 5 wt.-% | NaCl + | | |
| | Peach | fibre + | 5 wt.-% | 10 wt.-% | |
| | mix | 5 wt.-% pea | CaCl₂ | Soy protein | Tea |
| Substance [g] | [g] | NaHCO₃ [g] | [g] | [g] | extract |
| Microcrystalline | — | — | — | — | — |
| cellulose (MCC), | | | | | |
| Vivapur 105 | | | | | |
| Powdered glucose | — | — | — | — | — |
| syrup from corn | | | | | |
| DE43-47 | | | | | |
| Gummi Arabicum, | 200 | 200 | 200 | 200 | 200 |
| type Senegal | | | | | |
| Maltodextrin from | 500 | 500 | 500 | 500 | 500 |
| corn DE17-20 | | | | | |
| Mannitol D | 100 | — | — | — | — |
| NaCI, table salt | — | — | 50 | — | — |
| Whey protein conc. | — | — | — | — | — |
| 82% | | | | | |
| Peach flavour | 200 | 200 | 200 | 200 | 200 |
| Water | 1025 | 1300 | 900 | 1050 | 1000 |
| Calcium lactate | — | — | — | — | — |
| pentahydrate | | | | | |
| (CaLac) | | | | | |
| Sodium carbonate, | — | — | — | — | — |
| Na₂CO₃ | | | | | |
| Calcium chloride, | — | — | 50 | — | — |
| CaCl₂ | | | | | |
| Microcrystalline | — | — | — | — | — |
| cellulose (MCC), | | | | | |
| Vivapur 200 | | | | | |
| Pea fibre | — | 50 | — | — | — |
| Magnesium stearate | — | — | — | — | — |
| Inulin | — | — | — | — | — |
| Guar Gum | — | — | — | — | — |
| Trisodium citrate | — | — | — | — | — |
| Bamboo fibre | — | — | — | — | — |
| Apple pectin | — | — | — | — | — |
| Sodium hydrogen | — | 50 | — | — | — |
| carbonate, NaHCO₃ | | | | | |
| Soy protein | — | — | — | 100 | — |
| Tea extract | — | — | — | — | 100 |
| Total | 2025 | 2300 | 1900 | 2050 | 1900 |
| Viscosity [mPas] | 104 | 133 | 110 | 110-120 | 113 |
| D [3,2] [μm] | 5.557 | 4.35 | 5.55 | 5.85 | — |
| d10 [μm] | 3.823 | 2.93 | 3.81 | 3.76 | 3.43 |
| d50 [μm] | 12.294 | 8.6 | 12.64 | 12.85 | 12.1 |
| d90 [μm] | 28.099 | 22.91 | 34.37 | 48.03 | 33.53 |
| Water content [%] | 3.18 | 3.38 | 4.06 | 3.13 | 3.28 |
| Minimum ignition | 10 | 300 | 300 | 30 | 1000 |
| energy (MIE) [m]] | | | | | |
| 1. Burning rate | 20 | — | — | 30 | 23 |
| [sec] | | | | | |
| 2. Burning rate | 21 | — | — | 32 | 26 |
| [sec] | | | | | |
| Burning rate, mean | 21 | local | short | 31 | 25 |
| value [sec] | | burning | burning + | | |
| | | and sub. | rapid | | |
| | | extinguishing | extinguishing | | |

TABLE 5
Direct comparison of the minimum ignition energy (MIE) and burning rate of a powdered
peach mix comprising the fireproofing agents according to the invention (summary).

| | | | | | | | |
|---|---|---|---|---|---|---|---|
| Extracts | Black tea extract | 1000 | +++ | 23 | 26 | 25 | + |
| Combinations | 5% pea fibre + | 300 | +++ | − | − | local burning and | ++ |
| | 5% NaHCO₃ | | | | | sub. extinguishing | |
| | MCC (fine) + | 300 | +++ | − | − | local burning + | ++ |
| | Trisodium citrate | | | | | sub. extinguishing | |
| Fibres | Pea fibre | 100 | ++ | local burning | − | local burning | ++ |
| | Bamboo fibre | 30 | + | 48 | 47 | 48 | + |
| Dietary | Guar Gum | 100 | ++ | 22 | 25 | 24 | + |
| fibres | Apple pectin | 300 | +++ | 30 | 31 | 31 (local burning) | + |
| | Inulin | 100 | ++ | 21 | 19 | 20 | ++ |
| Proteins | Plant soy | 30 | + | 30 | 32 | 31 | + |
| | protein | | | | | | |
| | Animal: whey | 30 | + | 27 | 34 | 31 | + |
| | protein | | | | | | |
| Salts | Na₂CO₃ | 1000 | ++++ | short | short | short | ++ |
| | | | | scorching | scorching | scorching | |
| | CaLac | 100 | ++ | 25 | 25 | 25 | + |
| | 5% NaCl + | 300 | +++ | − | − | short burning + | ++ |
| | 5% CaCl₂ | | | | | rapid extinguishing | |
| | CaCl₂ | 100 | ++ | short | short | short | ++ |
| | | | | scorching | scorching | scorching | |
| | NaCl | 300 | +++ | 19 | 18 | 19 | + |
| | Trisodium citrate | 1000 | ++++ | − | − | no burning | +++ |
| | Magnesium | 30 | + | 29 | 30 | 30 | + |
| | stearate | | | | | | |
| Polysacch./ | Mannitol | 10 | +/− | 21 | 27 | 24 | + |
| sugar alcohols | Glucose | 10 | +/− | 23 | 21 | 22 | + |
| Insoluble | MCC 200 | 100 | ++ | 26 | 27 | 27 | + |
| Carbo- | (Ø 190 μm) | | | | | | |
| hydrates | MCC 105 | 30 | + | 26 | 27 | 27 | + |
| | (Ø 20 μm) | | | | | | |

| | | | | | | |
|---|---|---|---|---|---|---|
| Without fireproofing agent | 10 | reference | 20 | 21 | 21 | reference |

| | | | |
|---|---|---|---|
| MIE [mJ] | 1. Burning | 2. Burning | Burning rate, mean |

| | | |
|---|---|---|
| rate [sec] | rate [sec] | value [sec] |

* Improvement of the MIE:
“+” (improvement by one energy level) to “++++” (significant improvement of the MIE)
No improvement of the MIE: “+/−”
Improvement of the burning rate, i.e. increased burning rate: “+” (slight improvement) to “+++” (strong improvement)
No improvement of the burning rate: “+/−”

As can be seen from the above results, the finely powdered product formulations comprising the fireproofing agents according to the present invention show significantly improved MIE values corresponding to low reactivities and thus increased resistances to external ignition although exhibiting extremely low particle sizes. Consequently, the addition of an effective amount of the fireproofing agents according to the present invention advantageously reduces the hazardousness oft the corresponding powdered product formulations. Said additives as specified herein, prevent the dust explosion risk when said powdered compositions are manufactured, handled, transported, processed, or incorporated into a consumer product.

According to current knowledge, it is generally assumed that the MIE decreases with decreasing particle size and decreasing humidity. However, as shown above, it was surprisingly found that the fireproofing agents according to the present invention allow for relatively low water contents as well as small particle sizes while simultaneously allowing for a significant increase of the MIE. In addition, a comparison of the first column and the fourth column shows that an increase of the humidity, i.e. the water-content of the powdered composition does not lead to the desired effect.

In addition, all samples resulted in an improved burning rate, whereby the critical value could be reached for some substances such as for CaCl₂) or pea fibre as fireproofing additives. Here, only local burning or brief burning occurred, but not the entire powder section burned. Fibres and salts thus show good results here, which could certainly be controlled via the concentration.

Interestingly, CaCl₂) shows considerably improved MIE-values and burning rates although a corresponding sample shows a high burning number indicating fast spreading (see Example 1). Consequently, it might be concluded that there is no direct correlation between MIE, burning rate and burning number.

Surprisingly, it was found that such combinations advantageously improve the minimum ignition energy (MIE) and the burning rates of powdered compositions. In particular, it was found that only local burning occurred and/or that no fire propagation took place indicating a synergistical effect based on the combination of two or more different fireproofing agents.

TABLE 6
Burning rates of a powdered peach mix comprising the
fireproofing agents according to the invention.

| Peach flavour plus different | | | Burning |
|---|---|---|---|
| fireproofing agents (in powdered | 1. Burning | 2. Burning | rate, mean |
| form) | rate [sec] | rate [sec] | value [sec] |
| Peach mix + 10 wt.-% Pea fibre | 41 | 39 | 40 |
| Peach mix + 20 wt.-% Pea fibre | 51 | 53 | 52 |
| Peach mix + 5 wt.-% | 40 | 43 | 41.5 |
| NaCl + 5 wt.-% CaCl₂ | | | |
| Peach mix + 10 wt.-% | local burning + sub. extinguishing | | |
| NaCl + 10 wt.-% CaCl₂ | | | |

The samples were prepared according to the formulations specified above in Table 2. As can be seen from Table 6, combinations of two different fireproofing agents according to the present invention, such as two different inorganic salts result in a considerable improvement of the safety of an exemplary powdered food formulation in a synergistic manner compared to the single substances (here single salts).

TABLE 7
Comparison of the minimum ignition temperature of a
powdered peach mix comprising the fireproofing agents
according to the invention in a BAM-oven in ° C.

| Additional fireproofing agent | Minimum ignition |
|---|---|
| according to the invention | temperature was [° C.] |
| Without fireproofing agent | 350 |
| (“peach mix”) [reference] | |
| MCC 200 (Ø 190 μm) | 440 |
| NaCl | 410 |
| CaCl₂ | 360 |
| Apple pectin | 410 |
| Black tea extract | 470 |

The minimum ignition temperature was measured in a 170 mm long electrically heated and horizontally arranged pipe-oven which is called BAM-oven for the corresponding combustible dust/air mixtures. The required ignition temperature is measured at a vertically arranged impact plate in the centre of the oven for a dust sample vertically blown with air against said impact plate as specified in VDI 2263. The minimum ignition temperature is defined as the lowest temperature of a hot surface, determined under prescribed test conditions, at which the most ignitable air/dust mixture ignites, i.e. the ignition behaviour of swirled up dust on hot surfaces in a closed temperature field. To determine the ignition temperature the dust to be investigated is blown against a vertical impact surface—the hottest point inside the horizontally arranged furnace. The concentration of the dust/air mixture is changed by inputting different amounts of dust and the lowest temperature at which ignition occurs is determined. (pressure: 1 bar intermittent; concentration: 2 ml; verification with 1 and 4 ml).

The beverage formulation “peach mix” does not contain an additional fireproofing agent and serves as comparative example, i.e. the first column of the table shows the original recipe/composition. In the following columns, the mannitol was replaced by other components in the same proportion. The samples were prepared according to the formulations specified above in Table 2. As can be seen from Tables 4 and 5, all fireproofing agents according to the present invention allow for the improvement (i.e. increase) of the minimum ignition temperature of the analysed dust/air mixtures and thus for the preparation of more stable, i.e. less dangerous powdered compositions.

### Example 3: Taste of Different Powdered Compositions

In the following example the taste of different food product formulations comprising the fireproofing agents according to the present invention were evaluated.

TABLE 8
Taste evaluation.

| | Additional fireproofing agent according to Example 2 | | | |
|---|---|---|---|---|
| | Original “peach | | | |
| | mix” without | | | |
| | fireproofing | Bamboo | NaCl + | Trisodium |
| | agent | fibre | CaCl₂ | citrate |
| Subject | reference | slightly mealy | slight off- | slightly duller, |
| 1 | | | taste, watery | less fresh |
| Subject | reference | similar to the | slightly salty | slightly acidic |
| 2 | | original | | |
| Subject | reference | slightly more | slightly salty | similar to the |
| 3 | | bitter | | original |

The beverage formulation “peach mix” does not contain an additional fireproofing agent and serves as comparative example/as reference, i.e. the first column of the table shows the original recipe/composition. In the following columns, the mannitol was replaced by other components in the same proportion. The samples were prepared according to the formulations specified above in Table 2. As can be seen from Table 8, the fireproofing agent according to the present invention do not considerably change the taste/flavour of the powdered beverage formulation. In particular, salt-based fireproofing agents according to the present invention are highly suitable for use in salty food compositions such as salty snacks, while acid-derived fireproofing agents are more suitable for acidic food preparations and simultaneously are suitable to substitute additional salty or acidic components of the product formulations, respectively. In view of other consumer products such as cosmetic products, pharmaceutical products, homecare products, textile care products and the like all of the fireproofing agents according to the present invention can advantageously be added to the corresponding powdered compositions.

### Example 4: Evaluation of the Porosity, the Dust Index and the Flow Index of Spray-Dried Formulations Comprising Different Potential Fireproofing Agents

In order to analyse the flow behaviour of product formulations comprising different fireproofing agents according to the present invention, an aroma formulation (“peach mix”) as specified in Table 2 was provided (see Example 2).

The different samples were analysed by bulk density measurement and pycnometric density measurement. Based on these data the porosity of the samples can be determined according to the following formulas:

$$Interparticle ( interspace ) porosity = 1 - \frac{bulk density}{a p p arent particle density}$$

$$Intraparticle porosity = 1 - \frac{a p p arent particle density}{true particle density}$$

$$Total porosity = 1 - \frac{bulk density}{true particle density}$$

The value represents the proportion of pores between the particles. In other words, exactly the interstitial spaces and thus the “looseness” of the bulk. This can be an indication of how easily the sample can be fluidized.

The stresses acting on the powder during flow or in a static state in the production lines/silos etc. are determined by the powder's own weight, its bulk density (also poured density). The bulk density is measured according to common procedures. A defined volume of powder is filled in a container without applying compression and is subsequently weighed. This gives the weight per unit volume. The bulk density is calculated from the ratio of the mass of a bulk material to its bulk volume poured under defined conditions (in kg/m³). A free-flowing substance is generally incompressible so that its density will increase only slightly with increasing stress. In comparison, a very cohesive, poorly flowing bulk material will show a very large increase in bulk density with increasing stress.

Pycnometric density is determined using a gas pycnometer, i.e. a measuring instrument for determining the density of solids (=true particle density). Density is calculated as the ratio of mass to volume. The measuring principle in case of a powder is based on the displacement of air between and inside the powder particles by a pure gas, in the present case Helium. Depending on the particle structure, it is assumed that the helium does not reach the deeper cavities in the particle. In this case the determined density is referred to as the “apparent density”.

Generally, the true particle density is defined as the mass of the particle divided by its volume excluding open and closed pores and is the density of the solid material of which the particle is made. The apparent particle density is defined as the mass of a particle divided by its volume, excluding only the open pores and the effective particle density refers to the mass of a particle divided by its volume, including both open and closed pores.

The dust index (dust sedimentation analysis) is defined and was measured as follows: The powder samples were each analysed with the DustMon RD 100 instrument at room temperature. The instrument reliably measures the dustiness of granular products and powders, i.e. the amount of dust, and to determine the dust index. For this purpose, the sample (5 g) is placed on a flap, i.e. poured into the sample beaker. This flap opens at the start of the measurement so that the powder falls down a column, i.e. the sample drops down the tube into the sample collector. On the lower end of the device there are LED lights and sensors, which measure the shading of the sensors by the created dust over a certain period of time, i.e. the sedimentation time of the dust after impact on the bottom of the device. A curve is generated from this over approx. 30 seconds and a sum of the maximum value and the value after 30 seconds is calculated from this (dust concentration versus time). This quotient is defined as the dust index.

The flow index is defined and was measured as follows: The flow index was measured using a Texture Analyser (Powder Flow Analyser from Winopal GmbH). Based on a cohesion test the sample was analysed. With this test, the flow behaviour of powders can be objectively evaluated in comparison to other powders. During the actual test, the powder sample is “lifted” as it moves out of the sample, so that it slides over the rotor. The load cell, which is installed in the base of the Powder Flow Analyzer, records this change and uses the sample weight and the work performed to determine a statement about the flow behaviour of the powder. From the data thus determined, the flow index is obtained, which is evaluated as follows:

Flow index: Flow behaviour:

| | |
|---|---|
| <11 | free flowing |
| 11-14 | easy flowing |
| 14-16 | cohesive |
| 16-19 | very cohesive |
| >19 | extremely cohesive |

Flowability is a parameter for determining the flow behaviour of bulk solids and refers to the ability of bulk solids to flow vertically under defined conditions, i.e. the extent of free mobility or flow behaviour of bulk materials. In simpler terms, flowability is the property of powders to flow vertically. The flowability of a bulk material is determined by its composition and the nature of its particles. Influencing variables are, for example, particle size, particle size distribution, surface condition of the grains, adhering moisture.

The flowability is often specified as the run-out time of a defined sample quantity from defined hoppers and was determined based on the procedure as specified above. This flowability is denoted as “flow index”.

It was surprisingly found that the additives according to the present invention reduce the interstitial spaces of the powdered compositions. It was not possible to make this correlation apparent beforehand by means of the parameters flowability and dust index. This could explain physically why the substances increase the minimum ignition energy (MIE).

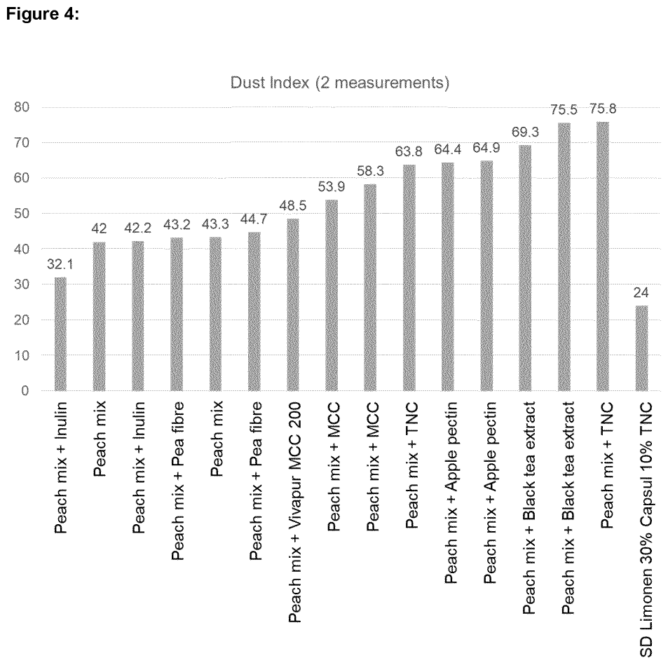

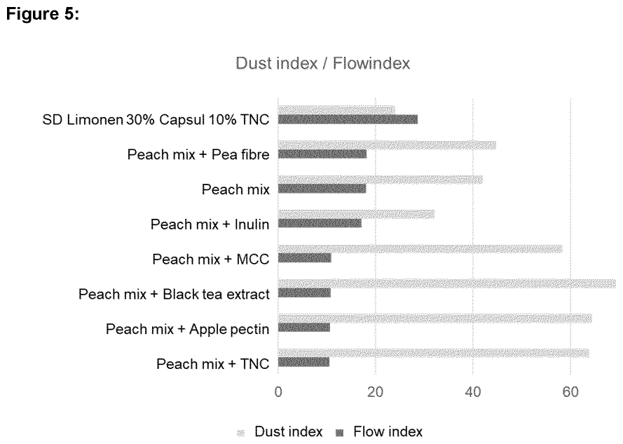

The sample “SD Limonene” is a reproduction of the patent US 2011/044922 A1 and serves as a reference. More specifically, the composition is as follows: 30% limonene, 60% Capsul (modified OSA starch), 10% trisodium citrate (=fireproofing agent). The sample has a dust value of 24. This is significantly lower compared to the samples according to the present invention. In addition, the reference shows a flow index of approximately 29 which is higher than the flow index of the sample according to the present invention. This higher flow index indicates that the reference sample is significantly more cohesive and less free flowing (a lower value indicates increased flowability). The interparticle porosity is even higher compared to the samples according to the present invention (see FIGS. 4 and 5 and Table 9, and “peach mix” (=sample without fireproofing agent)). Consequently, the minimum ignition energy is even lower compared to the samples according to the present invention (and “peach mix”). Thus, despite the addition of trisodium citrate, the sample is ignitable at very low energies.

However, it seems that the addition of plant gums and other high molecular weight substances show a high energy consumption during ignition (strong endothermic transition).

This effect was also found for fireproofing agents such as polysaccharides, sugar alcohols, proteins, water-soluble and/or water-insoluble dietary fibres, or mixtures thereof, and as specified herein.

It was surprisingly found that the addition of such substances to an existing highly flammable formulation “defuses” it by having the additives adsorb the energy for just these endothermic transitions upon ignition, thus making it no longer fully available for ignition.

TABLE 9
Dust index, flow index and bulk density of a powdered peach mix comprising
the fireproofing agents (10 wt %) according to the invention.

| | | Peach mix + | | | Peach mix + | Peach mix + | Peach mix + | SD Limonen |
|---|---|---|---|---|---|---|---|---|
| | Peach | Vivapur MCC | Peach mix + | Peach mix + | Apple pectin | Black tea | Vitacel Pea | 30% Capsul |
| | mix [g] | 200 [g] | MCC [g] | Inulin [g] | [g] | extract | fibre EF150 [g] | 10% TNC = |
| Water | 1025 | 1100 | 1145 | 1025 | | | | reference |
| Viscosity | 104 | 117 | 126 | 122 | 1350 | 1000 | 2150 | 35 |
| [mPas] | | | | | 120 | 113 | 138 | 61 |
| D [3,2] [μm] | 5.557 | 7.28 | 8.595 | 5.85 | | | | |
| d10 [μm] | 3.8 | 4.7 | 5.6 | 4.1 | 5.52 | 5.399 | 6.29 | 6.469 |
| d50 [μm] | 12.3 | 18.4 | 21.9 | 13.2 | 3.6 | 3.4 | 4.8 | 4.6 |
| d90 [μm] | 28.1 | 65.4 | 57.2 | 31.6 | 13.0 | 12.1 | 13.2 | 17.8 |
| Water content | 3.2 | 2.7 | 2.6 | 3.6 | 29.8 | 33.5 | 39.3 | 36.0 |
| [%] | | | | | 3.3 | 3.3 | 3.9 | 3.8 |
| MIE [mJ] | 10 | 100 | 30 | 100 | | | | |
| 1. Burning rate | 20 | 26 | 26 | 21 | 300 | 1000 | 100 | <10 |
| [sec] | | | | | 30 | 23 | Local | |
| 2. Burning rate | 21 | 27 | 27 | 19 | | | burning | |
| [sec] | | | | | 31 | 26 | | |
| Burning rate, | 20.5 | 26.5 | 26.5 | 20 | | | | |
| mean value | | | | | 31 | 25 | Local | 95 seconds per |
| [sec] | | | | | | | burning | 100 mm (1.05 |
| MIE dust (BAM- | 350 | 440 | | | | | | mm/second) |
| oven) | | | | | 410 | 470 | | |
| [° C.] | | | | | | | | |
| Dust index 1 | 42.0 | 48.5 | 53.9 | 32.1 | | | | |
| Dust index 2 | 43.3 | — | 58.3 | 42.2 | 64.9 | 75.5 | 43.2 | 24.00 |
| Density [g/cm³] | 1.121 | 1.054 | 1.050 | 1.124 | 64.4 | 69.3 | 44.7 | — |
| Bulk density | 0.381 | | 0.431 | 0.408 | 1.156 | 1.096 | 1.259 | 1.044 |
| [g/cm³] | | | | | 0.459 | 0.375 | 0.457 | 0.318 |
| Flow index | 18.10 | | 10.89 | 17.06 | | | | |
| Halogen dryer | 94.39 | 92.19 | 95.21 | 94.77 | 10.67 | 10.80 | 18.21 | 27.14 |
| 140° C. [%] | | | | | 95.24 | 95.27 | 93.72 | 96.17 |
| Halogen dryer | | | | | | | | |
| 140° C. 2 [%] | | | | | 95.04 | | | |
| Total porosity | 0.660 | — | 0.590 | 0.637 | | | | |
| | | | | | 0.603 | 0.658 | 0.637 | 0.695 |

The results of Table 9 show that all substances specified in the present invention are suitable fireproofing agents. The corresponding agents were added in amounts of 10 wt %. All samples according to the invention show improved MIE values compared to the reference (<10 for SD Limonen) as well improved dust index values although the samples according to the present invention show increased flowabilities. The flow index is inversely proportional to the actual flowability. That is, the more flowable the product, the smaller the flow index. Despite the low densities fireproofing was successfully achieved.

Although the additives make the powders more flowable and dusty (which would actually speak for an increased ignitability) surprisingly it appears that the apparent density might actually be reduced by the additives. The particle size of all samples is in a very similar range. Despite this the powders of the invention are less ignitable.

Particularly good results were achieved for powdered compositions further comprising an effective amount of polysaccharides, sugar alcohols, proteins, water-soluble and/or water-insoluble dietary fibres, plant-based oils and fats, salts of fatty acids having a chain length of 13 or more carbon atoms such as magnesium stearate, plant extracts such as tea extracts, or mixtures thereof, as the fireproofing agent/component.

### Example 5: Thermogravimetric Analysis

In addition, a standard formulation (“peach mix”=sample without fireproofing agent) and formulations with the fireproofing agents according to the present invention were analysed by means of thermogravimetric analysis (TGA device of Netzsch Instruments).

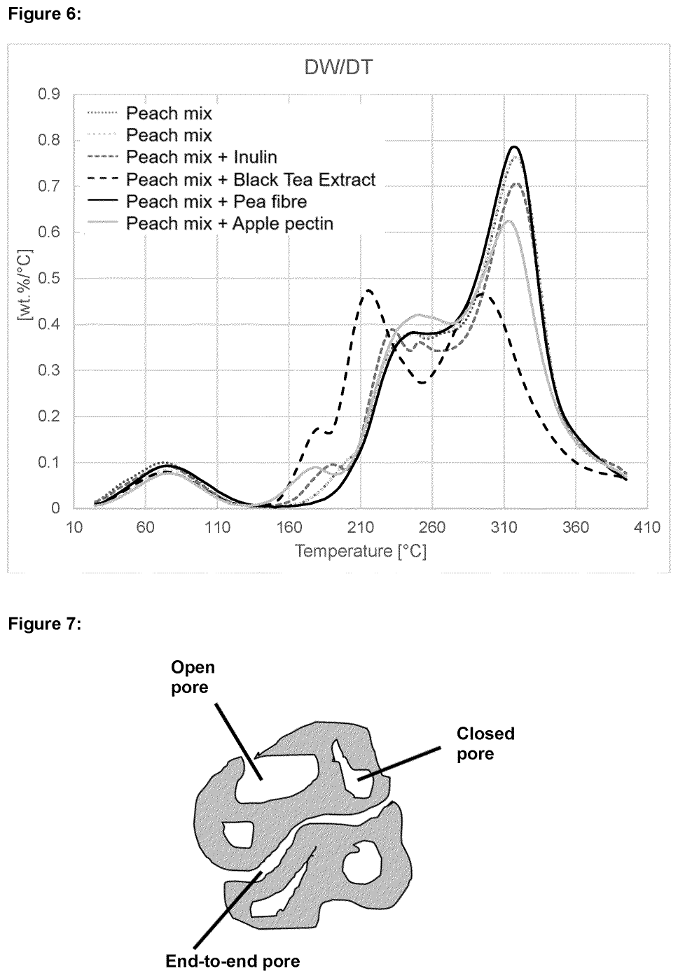

The results of the thermogravimetric analysis are shown in FIG. 6 (change of weight with change of temperature).

Two measurements were performed for a standard formulation (“peach mix”=sample without fireproofing agent) and prove the reproducibility of the method. The percent weight loss per degree of temperature was plotted showing the temperature at which the total formulation decomposes, and the degree of decomposition. The decomposition and thus the transition to the gaseous state is considered to be the preliminary stage of ignition.

The measurement shows that samples with apple pectin and inulin and also with tea extract show thermal decomposition earlier than the standard formulation (“peach mix”=sample without fireproofing agent). Energy is therefore consumed for decomposition, but this obviously does not contribute to ignition, as these formulations ignite significantly worse compared to the standard (see Table 4 and Table 9). The tea extract consists of different fractions with different molecular weights and structures, so that several decomposition peaks are recognizable here. This seems particularly effective according to the MIE data. The more complex the added substance, the later the decomposition occurs, which can be seen in the fibrous materials. If the decomposition occurs after that of the actual sample, the effect on the MIE does not seem to be quite as pronounced, as the cellulose (MCC) sample indicates. However, an effect is still present.

This leads to the conclusion that the decomposition behaviour of the additives influences the consumption of available energy during ignition.

1. A powdered composition comprising either
a spray-drying product, or
a composition comprising at least one microcapsule, and
optionally a carrier system, and
further comprising an effective amount of a fireproofing agent selected from the group consisting of polysaccharides, sugar alcohols, proteins, water-soluble and/or water-insoluble dietary fibres, plant-based oils and fats, salts of fatty acids having a chain length of 13 or more carbon atoms, plant extracts, and mixtures thereof;
wherein the powdered composition has a minimum ignition energy (MIE) of at least 10 mJ.

2. The powdered composition according to claim 1, wherein the fireproofing agent is selected from the group consisting of magnesium stearate, polysaccharides, sugar alcohols, proteins, water-soluble and/or water-insoluble dietary fibres, plant-based oils and fats, plant extracts, and mixtures thereof.

3. The powdered composition according to claim 1, wherein the powdered composition comprises from 1% to 80% by weight of the fireproofing agent(s) based on the total weight of the powdered composition.

4. The powdered composition according to claim 1, wherein the fireproofing agent comprises:
a mixture of at least one mineral salt with at least one salt of an organic acid,
a mixture of at least one polysaccharide, at least one sugar alcohol, at least one protein, at least one water-soluble and/or water-insoluble dietary fibre, and/or at least one plant extract with at least one salt of an organic acid, or
a mixture of at least one polysaccharide, at least one sugar alcohol, at least one protein, at least one water-soluble and/or water-insoluble dietary fibre, and/or at least one plant extract with at least one mineral salt.

5. The powdered composition according to claim 1, wherein the powdered composition comprises at least one perfuming and/or flavouring active ingredient.

6. The powdered composition according to claim 1, wherein the powdered composition particle size D50 is 50 μm or less.

7. The powdered composition according to claim 1, wherein the powdered composition has a burning rate of ≤2.2 mm/s.

8. The powdered composition according to claim 1, wherein the powdered composition has a minimum ignition temperature of a whirled-up dust cloud of ≥300° C.

9. Method for the preparation of a powdered composition comprising the following steps:
(a) providing an aqueous dispersion, optionally comprising microcapsules, comprising a fireproofing agent selected from the group consisting of polysaccharides, sugar alcohols, proteins, water-soluble and/or water-insoluble dietary fibres, plant-based oils and fats, salts of fatty acids having a chain length of 13 or more carbon atoms, plant extracts, and mixtures thereof, and
(b) spray-drying the dispersion of step (a), to obtain the powdered composition.

10. The method according to claim 9, wherein the aqueous dispersion comprises at least one microcapsule or wherein the product of step (b) is a spray-drying product.

11. The method according to claim 9, wherein the dispersion of step (a) comprises an additional carrier.

12. The method according to claim 9, further comprising the step of:
(c) agglomerating the powdered composition after step (b).

13. The powdered composition prepared according to the method of claim 9, wherein the powdered composition comprises ≤6% by weight of remaining water based on the total weight of the powdered composition.

14. A method of reducing the combustion potential of a powdered composition comprising formulating the composition with a fireproofing agent selected from the group consisting of polysaccharides, sugar alcohols, proteins, water-soluble and/or water-insoluble dietary fibres, plant-based oils, salts of fatty acids having a chain length of 13 or more carbon atoms, plant extracts, and mixtures thereof for the preparation of a powdered composition.

15. Consumer product comprising a powdered composition according to claim 1.

16. The composition according to claim 1, wherein the salt of a fatty acid having a chain length of 13 or more carbon atoms is magnesium stearate, and the plant extracts are tea extracts.

17. The composition according to claim 1 wherein the MIE is at least 300 mJ.

18. The composition according to claim 3 wherein the composition comprises from 10% to 40% by weight of the fireproofing agent(s) based on the total weight of the powdered composition.

19. The composition according to claim 6, wherein the particle size D50 is 20 μm or less.

20. The composition according to claim 8 wherein the composition has a minimum ignition temperature of a whirled-up dust cloud of ≥420° C.
